(19) United States
(12) Patent Application Publication
(10) Pub. No.: US 2005/0148556 A1
(43) Pub. Date: Jul. 7, 2005

(54) Compositions and methods for increasing HDL and HDL-2b levels

(76) Inventor: Raif Tawakol, Merced, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/977,508

(22) Filed: Oct. 29, 2004

Related U.S. Application Data
(60) Provisional application No. 60/515,891, filed on Oct. 29, 2003.

(51) Int. Cl. A61K 31/60; A61K 31/455; A61K 31/405; A61K 31/192; A61K 9/20

(52) U.S. Cl. 514/165; 514/356; 514/420; 514/569; 514/570; 424/464

(57) ABSTRACT

The present invention provides compositions and methods for reducing flushing in a patient. In addition, compositions and methods are provided for increasing HDL and/or HDL-2b levels in a patient. In some embodiments, the compositions include an adipocyte G-protein antagonist, a PPAR-α agonist, and a PPAR-γ agonist in amounts effective in to provide a synergistic therapeutic HDL increasing effect, and/or a synergistic therapeutic HDL-2b increasing effect.

### CROSS-REFERENCES TO RELATED APPLICATIONS

This application claims the benefit of U.S. Provisional Application No. 60/515,891, filed Oct. 29, 2003 which is herein incorporated by reference in its entirety for all purposes.

### BACKGROUND OF THE INVENTION

A cluster of inter-related plasma lipid and lipoprotein abnormalities associated with alterations in HDL (high density lipoprotein) and HDL-2b metabolism contributes to the risk of atherosclerosis and cardiovascular events in patients with insulin resistance and type 2 diabetes. HDL and HDL-2b levels control atherogenesis, vascular inflammation, endothelial function and thrombogenicity. The alteration in particle size of both HDL and LDL (low density lipoprotein) contribute to events and progression of disease. Therefore there is a need in the art for therapies that increase HDL and HDL-2b levels.

Niacin has been used in an attempt to raise HDL levels and to lower very low density lipoprotein (VLDL) triglycerides and LDL levels. When tolerated, it is effective as either primary therapy or adjunctive therapy. Numerous side effects limit its use in well over 50% of patients in which it is tried. These side effects include an intense inflammation, or flushing, and associate itching, or pruritus, that usually involves the face and upper part of the body, often involving the entire body.

While niacin has many beneficial properties, it also possesses at least two important side effects. First is hepatotoxicity. High doses of niacin have adverse effects on the liver. Cases of severe hepatic toxicity, including fulminant hepatic necrosis have occurred in patients who have substituted sustained-release (discussed below)(otherwise known as modified-release or timed-release) niacin products for immediate-release (crystalline) niacin at equivalent doses. The second important side effect is flushing. Niacin, even in low doses, stimulates the production of prostaglandins, which participate in the body's defenses against infection. Increased prostaglandin synthesis induces the production of the inflammatory cytokines, cyclooxygenase, and also plays a part in causing inflammation in the body. Thus, ingestion of niacin manifests itself in an increase in inflammation, also known as flushing.

Aspects of the present invention address these and other problems.

### BRIEF SUMMARY OF THE INVENTION

The present invention provides a completely new modality in the treatment of diabetes, insulin resistance, metabolic syndrome, hyperlipidemia, dyslipidemia, cardiovascular disease, atherosclerosis, and hypercholesterolemia. Surprisingly, the combination of an adipocyte G-protein antagonist, a peroxisome proliferator-activated receptor-α (PPAR-α) agonist, and a peroxisome proliferator-activated receptor-γ agonist (PPAR-γ) has been found to effectively increase levels of high density lipoproteins (HDLs) and/or HDL-2b levels. Moreover, it has been discovered that co-administration of an NSAID with an adipocyte G-protein antagonist over a period of less than 12 hours and not more than 4 hours provides a superior reduction of flushing in patients while reducing or eliminating symptoms of liver damage relative to previously known formulations.

In one aspect, the present invention provides a composition including a first amount of an adipocyte G-protein antagonist, a second amount of a peroxisome proliferator-activated receptor-α agonist, and a third amount of a peroxisome proliferator-activated receptor-γ agonist. The first amount, second amount, and third amount are together an effective amount to provide a synergistic therapeutic HDL increasing effect, and/or a synergistic therapeutic HDL-2b increasing effect.

In another aspect, an intermediate release solid unit dosage form is provided. The intermediate release solid unit dosage form includes a niacin, a nonsteroidal anti-inflammatory drug, and an intermediate release excipient. The niacin and the nonsteroidal anti-inflammatory drug are present in a single layer of the solid unit dosage. The niacin and nonsteroidal anti-inflammatory drug are provided in amounts effective to reduce flushing in a patient relative to the amount of flushing observed with niacin alone. The niacin and nonsteroidal anti-inflammatory drug may also be provided in amounts effective to increase HDL and/or HDL-2b levels.

In another aspect, a method is provided for treating hyperlipidemia, dyslipidemia, atherosclerosis, a hypercholesterolemia, cardiovascular disease, diabetes, insulin resistance, and/or metabolic syndrome in a patient in need of such treatment. The method includes administering to the patient a composition having a first amount of an adipocyte G-protein antagonist, a second amount of a PPAR-α agonist, and a third amount of a PPAR-γ agonist. The first amount, second amount, and third amount are together an effective amount to provide a synergistic therapeutic HDL increasing effect, and/or a synergistic therapeutic HDL-2b increasing effect.

In another aspect, a method is provided for reducing flushing in a subject receiving niacin. The method includes co-administering the niacin and a nonsteroidal anti-inflammatory drug to the subject over a period of less than 12 hours and more than 4 hours.

### DETAILED DESCRIPTION OF THE INVENTION

Abbreviations and Definitions

An “active agent” or “active ingredient” is a component of a dosage form, pharmaceutical composition, or composition of the present invention that performs a biological function when administered or induces or affects (enhances or inhibits) a physiological process in some manner. “Activity” is the ability to perform the function, or to induce or affect the process. Active agents and ingredients are distinguishable from excipients such as carriers, vehicles, diluents, lubricants, binders, and other formulating aids, and encapsulating or otherwise protective components. Active ingredients may also be referred to herein as a “component” of the compositions of the present invention.

A “synergistic therapeutic HDL increasing effect,” or “synergistic therapeutic HDL-2b increasing effect,” as used herein, means that a given combination of at least 3 compounds exhibits synergy when tested in an HDL or HDL-2b increasing assay (see Assays for Testing the HDL or HDL-2b Increasing Activity, below). “Synergy,” as described for example by Chou, et al., Adv Enzyme Regul 22: 27-55 (1984), occurs when the measured effect (in this case, an HDL or HDL-2b increasing effect) of the compounds when administered in combination is greater than the additive effect of the compounds when each is administered alone as a single agent. Chou, et al. provide an exemplary method of measuring synergy based on Michaelis-Menton kinetics where combination effects are reduced to a numeric indicator referred to as the combination index (C.I.). Where the combination index is less than 1, synergism is indicated. Where the combination index is equal to 1, summation (also commonly referred to as additivity) is indicated. Where the combination index is greater than 1, antagonism is indicated.

The abbreviation “HDL” refers to high density lipoprotein. The abbreviation “HDL-2b” refers to the gradient gel electrophoresis subclass of HDL having the 2b designation, which includes apoprotein A-I.

The phrase “therapeutically effective amount” means an amount sufficient to produce a therapeutic result. Generally the therapeutic result is an objective or subjective improvement of a disease or condition, achieved by inducing or enhancing a physiological process, blocking or inhibiting a physiological process, or in general terms performing a biological function that helps in or contributes to the elimination or abatement of the disease or condition.

A “subject” as used herein generally refers to any living multicellular organism. Subjects include, but are not limited to animals (e.g., cows, pigs, horses, sheep, dogs and cats) and plants, including hominoids (e.g., humans, chimpanzees, and monkeys). The term includes transgenic and cloned species. The term “patient” refers to both human and veterinary subjects.

The phrase “substantially homogeneous,” when used to describe a formulation (or portion of a formulation) that contains a combination of components, means that the components, although each may be in particle or powder form, are fully mixed so that the individual components are not divided into discrete layers or form concentration gradients within the formulation.

“Unit dosage form” refers to a composition intended for a single administration to treat a subject suffering from a disease or medical condition. Each unit dosage form typically comprises an active ingredient of this invention plus pharmaceutically acceptable excipients. Examples of unit dosage forms are individual tablets, individual capsules, bulk powders, and liquid solutions, emulsions or suspensions. Beneficial modification of the disease or condition may require periodic administration of unit dosage forms, for example: one or two unit dosage forms two or more times a day, one or two with each meal, one or two every four hours or other interval, or only one per day. The expression “oral unit dosage form” indicates a unit dosage form designed to be taken orally. A “solid unit dosage form” indicates a unit dosage form in solid state at the time of administration.

“Controlled” or “sustained” or “time release” delivery are equivalent terms that describe the type of active agent delivery that occurs when the active agent is released from a delivery vehicle at an ascertainable and manipulatable rate over a period of time, which is generally on the order of minutes, hours or days, typically ranging from about thirty minutes to about 3 days, rather than being dispersed immediately upon entry into the digestive tract or upon contact with gastric fluid. A controlled release rate can vary as a function of a multiplicity of factors. Factors influencing the rate of delivery in controlled release include the particle size, composition, porosity, charge structure, and degree of hydration of the delivery vehicle and the active ingredient(s), the acidity of the environment (either internal or external to the delivery vehicle), and the solubility of the active agent in the physiological environment, i.e., the particular location along the digestive tract. “Intermediate time release” or “intermediate release” refers to those formulations that release active agent from the delivery vehicle over a period of less than 12 hours and more than 5 hours. In an exemplary embodiment, the period of release is from about 5 to 9 hours. In another exemplary embodiment, the period is from 5 to 8 hours. In another exemplary embodiment, the period is from 6 to 8 hours. In another exemplary embodiment, the period about 7 hours.

The phrase “therapeutically effective amount” means an amount sufficient to produce a therapeutic result. Generally the therapeutic result is an objective or subjective improvement of a disease, achieved by inducing or enhancing a physiological process, blocking or inhibiting a physiological process, or in general terms performing a biological function that helps in or contributes to the elimination or abatement of the disease or condition.

Components of the compositions of the invention may be present as pharmaceutically acceptable salts. The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al. Journal of Pharmaceutical Science 66: 1-19 (1977)). Certain specific compounds of the present invention contain both basic and acidic functionalities that allow the compounds to be converted into either base or acid addition salts.

The neutral forms of the components are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides chemical compounds, such as niacin, NSAID, tryptophan, fibrates, thiazolidinediones, biguanides and/or the pharmaceutical excipients, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment.

Certain chemical compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain chemical compounds of the present invention possess asymmetric carbon atoms (optical centers) or double bonds; the racemates, diastereomers, geometric isomers and individual isomers are encompassed within the scope of the present invention. In an exemplary embodiment, niacin is a racemate. In another exemplary embodiment, niacin is substantially (over 70%) enantiomerically pure in one of the stereoisomers.

I. Compositions Including an Adipocyte G-Protein Antagonist, a PPAR-α Agonist, and a PPAR-γ Agonist

It has been discovered that, surprisingly, an adipocyte G-protein antagonist, a peroxisome proliferator-activated receptor-α (PPAR-α) agonist, and a peroxisome proliferator-activated receptor-γ agonist (PPAR-γ) may be combined to effectively increase levels of high density lipoproteins (HDLs) and/or HDL-2b levels. Due to the complimentary action of these three components, HDL levels and/or HDL-2b levels may be increased while minimizing undesired side effects of any one component. Thus, the combination may be used to increase HDL levels and/or HDL-2b levels in a wide variety of subjects, such as those with diabetes, insulin resistance, metabolic syndrome, hyperlipidemia, dyslipidemia, cardiovascular disease, atherosclerosis, and hypercholesterolemia. The combination may also be used to induce weight loss and/or a decrease levels of free fatty acids (including fatty acid esters) in a subject. In addition, it has been discovered that the adipocyte G-protein antagonist, PPAR-α agonist, and PPAR-γ agonist may be combined in amounts that are effective in providing a synergistic therapeutic HDL increasing effect and/or a synergistic therapeutic HDL-2b increasing effect.

In one aspect, the present invention provides a composition including a first amount of an adipocyte G-protein antagonist, a second amount of a peroxisome proliferator-activated receptor-α agonist, and a third amount of a peroxisome proliferator-activated receptor-γ agonist. The first amount, second amount, and third amount are together an effective amount to provide a synergistic therapeutic HDL increasing effect, or a synergistic therapeutic HDL-2b increasing effect.

In some embodiments, the first amount, second amount, and third amount further provide complimentary action between the adipocyte G-protein antagonist, PPAR-α agonist, and PPAR-γ agonist components such that HDL and/or HDL-2b levels are raised while minimizing undesired side effects of any one component.

For example, it is well known that niacin, an adipocyte G-protein antagonist, may increase blood sugar levels in subjects with early on-set diabetes thereby exacerbating the diabetic condition. See Wang et al., Am J Physiol Endocrinol Metab 279: E50-9 (2000). Therefore, although niacin may moderately increase HDL levels in a subject with early on-set diabetes, the fact that niacin increases blood sugar levels prevents the clinical application of niacin to the early on-set diabetic patient population. However, niacin may be combined with a PPAR-α agonist and a PPAR-γ agonist to decrease blood sugar levels in a subject with early on-set diabetes while effectively increasing HDL and/or HDL-2b levels. Thus, in some embodiments, the combination of a niacin, a PPAR-α agonist, and a PPAR-γ agonist provide a diabetes corrective effect.

Niacin has also been shown to raise blood sugar levels in individuals with metabolic syndrome and/or insulin resistance. See Grundy et al., Arch Intern Med 162: 1568-76 (2002). However, niacin may be combined with a PPAR-α agonist and a PPAR-γ agonist to effectively increase HDL and/or HDL-2b levels while not substantially increasing blood sugar levels in subjects with metabolic syndrome or insulin resistance. A blood sugar level that does not substantially increase in a subject with metabolic syndrome or insulin resistance means that the blood sugar level does not significantly increase the ratio of triglycerides to HDL or significantly decrease the body's response to insulin, respectively. In some embodiments, the blood sugar level does not increase more than about 1%, 0.1%, or 0.01% after administration of the adipocyte G-protein antagonist, PPAR-α agonist, and PPAR-γagonist combination.

Thus, in some embodiments, the composition that includes the combination of a PPAR-α agonist, a PPAR-γ agonist, and an adipocyte G-protein antagonist are combined in amounts effective to increase HDL and/or HDL-2b levels while minimizing side effects associated with any one component that may be detrimental to subjects having diabetes, insulin resistance, or metabolic syndrome. In related embodiments, the combination may additionally provide amelioration of diabetes, metabolic syndrome, or insulin resistance.

In addition to having beneficial properties for subjects with diabetes, metabolic syndrome, or insulin resistance, the combination may also increase HDL and/or HDL-2b levels while minimizing side effects of any one component of the combination that may be detrimental to subjects afflicted with cardiovascular disease, hyperlipidemia, atherosclerosis, or hypercholesterolemia. In some embodiments, the combination provides an HDL and/or HDL-2b increasing effect while additionally providing amelioration of cardiovascular disease, hyperlipidemia, dyslipidemia, an atherosclerosis, and/or a hypercholesterolemia.

Compositions having a combination of an adipocyte G-protein antagonist, a PPAR-α agonist, and a PPAR-γ agonist may be combined in amounts effective in providing a synergistic therapeutic HDL increasing effect, and/or a synergistic therapeutic HDL-2b increasing effect. Synergism is defined above and exemplary assays for determining synergy are provided below. In some embodiments, the components are combined in amounts effective in providing an HDL increasing effect of more than 40% in a subject relative to the HDL levels in the subject prior to treatment. In an exemplary embodiment, the HDL increasing effect is greater than 50%, 60%, 70%, 80%, 90%, 100%, 110%, 120%, 130%, 140%, 150%, 160%, 170%, 180%, 190%, or 200%. Exemplary ranges of HDL increases include from 50% to 300%, 60% to 250%, 70% to 200%, 80% to 175%, and 90% to 150%.

The HDL-2b increasing effect may be greater than 50%, 75%, 100%. 125%, 150%, 175%, 200%, 225%, 250%, 275%, 300%, 325%, 350%, 375%, 400%, 425%, 450%, 475%, or 500%. Exemplary ranges of HDL-2b increasing effects include from 50% to 600%, 100% to 500%, and 200% to 400%.

In addition to an adipocyte G-protein antagonist, a PPAR-α agonist, and a PPAR-γagonist, the composition may further include additional components. Useful additional components include non-steroidal anti-inflammatory drugs (“NSAIDs”). NSAIDs are discussed in more detail below in the context of niacin-NSAID combinations. The embodiments of niacin-NSAID combinations discussed below are equally applicable to the present compositions containing an adipocyte G-protein antagonist, a PPAR-α agonist, and a PPAR-γ agonist.

In some embodiments, the composition additionally includes a biguanide. Biguanides are discussed in more detail below. In an exemplary embodiment, the biguanide is metformin.

A wide variety of adipocyte G-protein antagonists, PPAR-α agonists and PPAR-γagonists are useful in the present composition. In an exemplary embodiment, the adipocyte G-protein antagonist is a niacin, the PPAR-α agonist is a fibrate, and the PPAR-γ agonist is a thiazolidinedione. In a related embodiment, the fibrate is a fenofibrate, and the thiazolidinedione is selected from rosiglitazone, pioglitazone, muraglitizone and farglitazar. In another related embodiment, where the thiazolidinedione is rosiglitazone, the composition additionally includes a biguanide, such as metformin.

A. Adipocyte G-Protein Antagonists

Adipocyte G-protein antagonists are compounds that inhibit cyclic adenosine monophosphate (cAMP) accumulation in adipose tissue through a G(i)-protein-mediated inhibition of adenylyl cyclase. See Tunaru et al., Nat Med. 9(3): 352-5 (2003). The primary action of adipocyte G-protein antagonists is to decrease lipolysis in adipose tissue by inhibiting hormone-sensitive triglyceride lipase. Niacin, an exemplary adipocyte G-protein antagonist, has been shown to bind to the mouse PUMA-G (protein upregulated in macrophages by interferon-gamma) and human HM74 resulting in a G(i)-mediated decrease in cAMP levels. Id.

Other characteristics of adipocyte G-protein antagonists may include decreased production of VLDL (Mahley et al., Williams Textbook of Endocrinology 9^{th} Edition, Chapter 23, p. 1143), which may be due, at least in part, to a transient inhibitory effect of niacin on lipolysis, a decreased delivery of free fatty acids to the liver, and a decrease in triglyceride synthesis and VLDL-triglyceride transport. Enhanced clearance of VLDL also may occur, possibly owing to enhanced activity of the lipoprotein lipase. The decrease in LDL levels could be due to decreased VLDL production and enhanced hepatic clearance of LDL precursors. Niacin also raises HDL cholesterol levels, decreases clearance rate of apoA-I, and decreases synthesis of apoA-II (Shephard et al., J. Clin. Invest. 63: 858-867 (1979)). Adipocyte G-protein antagonists typically do not alter the rates of cholesterol synthesis or bile acid excretion.

In an exemplary embodiment, the adipocyte G-protein antagonist is a niacin. The term “niacin,” as used herein, refers to nicotinic acid, nicotinic acid derivatives and prodrugs that function as adipocyte G-protein antagonists (e.g. acipimox), and all pharmaceutically acceptable equivalents and salts thereof (e.g. Niaspan®, Nicolar®, and the like). See also U.S. Pat. No. 6,677,361; Miller et al., Am. J. Clin. Nutr. 8: 480-490 (1960); and Neuvonen et al., Br. J. Clin. Pharmacol. 32: 473-476 (1991), which are herein incorporated by reference in their entirety for all purposes). The term “nicotinic acid” refers to a pyridine-3-carboxylic acid (i.e. vitamin B₃), including its salts and/or pharmaceutically acceptable equivalents.

B. PPAR-α Agonists

PPAR-α agonists are compounds that reduce accumulation of free fatty acids in muscle cells by activating the peroxisome proliferator-activated receptor (PPAR)-α and downregulating the acyl cholesteryl-2 (ACC-2) receptor. PPAR-α agonists do not substantially effect levels of adiponectin. It has been established that activation of PPAR-α results in transcription of enzymes that increase fatty acid catabolism and decrease de novo fatty acid synthesis in the liver resulting in decreased triglyceride synthesis and VLDL production/secretion. In addition, PPAR-α activation downregulates production of apoC-III, an inhibitor of LPL activity, thereby increases clearance of VLDL. See Auwerx et al. Atherosclerosis 124(Suppl.): S29-S37 (1996). Thus, administration of PPAR-α agonists may also result in one or more of the following effects: lowering serum triglycerides, lowering of LDL cholesterol levels in liver and fat cells, shifting the LDL particle size from the more atherogenic small dense to normal dense LDL, increasing HDL cholesterol, decreasing ApoC-III levels, increasing ApoC-II levels, and increasing ApoA-I levels. Additional characteristics and methods of assessing those characteristics are well known in the art, and are discussed in more detail in Torra et al., Curr Opin Lipidol 12: 245-254 (2001), and Henson, Proc. Nat'l. Acad. Sci. 100: 6295-6296 (2003).

In an exemplary embodiment, the PPAR-α agonist is a fibrate. See, Staels et al., Pharm. Des. 3(1): 1-14 (1997). Fibrates are a class of drugs which may lower serum triglycerides by 20-50%, lower LDL cholesterol by 10-15%, shift the LDL particle size from the more atherogenic small dense to normal dense LDL, and increase HDL cholesterol by 10-15%.

Fibrates useful in the present invention ureidofibrate as well as those listed in Table 1, including acceptable salts, prodrugs, and pharmaceutically acceptable equivalents thereof. The Patent Nos. listed in Table 1 are incorporated herein by reference in their entirety for all purposes.

TABLE 1

| FIBRATES | ALTERNATIVE NAME | PATENT NO. |
|---|---|---|
| Beclobrate | Beclipur; Turec | U.S. Pat. No. 4,483,999 |
| Bezafibrate | Benfizal; Benzalip; | U.S. Pat. No. 3,781,328 |
| | Bezatol; Cedur; Difaterol | |
| Binifibrate | | |
| Ciprofibrate | Ciprol; Lipanor; Modalim | U.S. Pat. No. 3,948,973 |
| Clinofibrate | Lipoclin | U.S. Pat. No. 3,716,583 |
| Clofibrate | Amotril; Anparton; Apolan; | U.S. Pat. No. 3,262,850 |
| | Artevil; Ateculon; Arteriosan; | |
| | Atheropront; Atromidin; | |
| | Atromid-S; Biosclercan; | |
| | Claripex; Clobren-SF; | |
| | Clofinit; CPIB; Hyclorate; | |
| | Liprinal; Neo-Atromid; | |
| | Normet; Normolipol; Recolip; | |
| | Regelan; Serotinex; Sklerolip; | |
| | Skleromexe; Sklero-Tablinen; | |
| | Ticlobran; Xyduril | |
| Clofibric Acid | | GB 860,303 |
| Etofibrate | | U.S. Pat. No. 3,723,446 |
| Fenofibrate | Ankebin; Elasterin; Fenobrate; | U.S. Pat. No. 4,058,552 |
| | Fenotard; Lipanthyl; Lipantil; | |
| | Lipidil; Lipoclar; Lipofene, | |
| | Liposit; Lipsin; Nolipax; | |
| | Procetoken; Protolipan; | |
| | Secalip | |
| Gemfibrozil | Decrelip; Genlip; Gevilon; | U.S. Pat. No. 3,674,836 |
| | Lipozid; Lipur; Lopid | |
| Nicofibrate | | U.S. Pat. No. 3,369,025 |
| Pirifibrate | | |
| Ronifibrate | Bratenol | U.S. Pat. No. 3,971,798 |
| Simifibrate | Cholesoivin; Liposolvin | U.S. Pat. No. 3,494,957 |
| Theofibrate | Duolip | U.S. Pat. No. 3,984,413 |

Other useful PPAR-α agonists include GW-641597 (GlaxoSmithKline), GW-590735 (GlaxoSmithKline), K-111 (Roche), and LY-518674 (Lilly).

In an exemplary embodiment, the fibrate is fenofibrate (C₂₀H₂₁ClO₄), including salts, prodrugs, and pharmaceutically acceptable equivalents thereof.

C. PPAR-γ Agonists

PPAR-γ agonists are compounds that are capable of increasing levels of adiponectin by activating the peroxisome proliferator-activated receptor (PPAR)-γ. Administration of PPAR-γ agonists may also result in one or more of the following effects: an increase in HDL levels, reduction in free fatty acid levels, mobilization of sugar in muscle cells, promotion of free fatty acid dispersion in the muscle compartment, reduction of VLDL in the liver, upregulation of cadherin receptors (including T-cadherin, N-cadherin, and L-cadherin), and an increase in the number of adipocytes. As used herein, a PPAR-γ agonist includes PPAR-γα agonists (also referred to herein as “dual receptor agonists”). Additional characteristics of PPAR-γ agonists and methods of assessing those characteristics are well known in the art, and are discussed in more detail in Torra et al., Curr Opin Lipidol 12: 245-254 (2001), and Henson, Proc. Nat'l. Acad. Sci. 100: 6295-6296 (2003).

In an exemplary embodiment, the PPAR-γ agonist is a thiazolidinedione (also known as a glitazone), or a pharmaceutical composition or salts thereof. Thiazolidinediones (“TZDs”) have been used in the treatment of diabetes. Useful PPAR-γ agonists include, for example, those described in U.S. Pat. Nos. 6,673,815 and 6,670,380, which are herein incorporated by reference in their entirety for all purposes. In an exemplary embodiment, the PPAR-γ agonist is selected from troglitazone (Wamer-Lambert's Rezulin®, disclosed in U.S. Pat. No. 4,572,912), rosiglitazone (SKB), pioglitazone (Takeda), Mitsubishi's MCC-555 (disclosed in U.S. Pat. No. 5,594,016), Glaxo-Welcome's GL-262570, englitazone (CP-68722, Pfizer), darglitazone (CP-86325), Pfizer, isaglitazone (MIT/J&J), JTT-501 (JPNT/P&U), L-895645 (Merck), R-119702 (Sankyo/WL), NN-2344 (Dr. Reddy/NN), ragaglitazar, YM-440 (Yamanouchi), AZ-242/tesaglitazar (Astra/Zeneca; as described: in B. Ljung et. al., J. Lipid Res., 2002, 43, 1855-1863), AR-HO39242 (Astra/Zeneca), GW-409544 (Glaxo-Wellcome), KRP297 (Kyorin Merck), those disclosed by Murakami et al, “A Novel Insulin Sensitizer Acts As a Coligand for Peroxisome Proliferation—Activated Receptor Alpha (PPARα) and PPARγ Effect on PPARα Activation on Abnormal Lipid Metabolism in Liver of Zucker Fatty Rats”, Diabetes 47: 1841-1847 (1998), LY-674 (Lilly), LYH-929 (Lilly), GW-409544 (Glaxo-Wellcome), DRF-4832 (Dr. Reddy's), MK-0767 (Merck), muraglitazone (BMS), farglitazar, and TZD18 (Merck).

In some embodiments, the PPAR-α agonist is selected from muraglitizone, farglitazar, rosiglitazone and pioglitazone.

D. Biguanides

The term “biguanide,” as used herein, refers to compounds that inhibit hepatic glucose production and increase the sensitivity of peripheral tissues to insulin without increasing pancreatic insulin production. Biguanides prevent the desensitization of human pancreatic islets usually induced by hyperglycemia with little or no significant effect on the secretion of glucagon or somatostatin. In some embodiments, the biguanide does not significantly increase lactate production from skeletal muscle (lactic acidosis).

Exemplary biguanides include metformin, phenformin, buformin, prodrugs and pharmaceutically acceptable salt thereof (e.g. Glucophage®, metformin hydrochloride or the metformin salts described in U.S. Pat. Nos. 3,957,853, 4,080,472, 6,693,094, and 6,790,45. which are herein incorporated by reference in their entirety for all purposes).

In an exemplary embodiment, the biguanide is metformin. Glucose levels are reduced during metformin therapy secondary to reduced hepatic glucose output from inhibition of gluconeogenesis and glycogenolysis. Metformin also may decrease plasma glucose by reducing the absorption of glucose from the intestine, but this does not appear to be of clinical importance. Improved insulin sensitivity in muscle from metformin may be derived from multiple events, including increased insulin receptor tyrosine kinase activity, augmented numbers and activity of GLUT4 transporters, and enhanced glycogen synthesis.

Metformin clinically decreases plasma triglyceride and low-density lipoprotein (LDL) cholesterol levels by 10% to 15%, reduces postprandial hyperlipidemia, decreases plasma free fatty acid levels, and free fatty acid oxidation. HDL cholesterol levels either do not change or increase slightly after metformin therapy.

II. Compositions Including an Adipocyte G-Protein Antagonist and Non-Steroidal Anti-Inflammatory Drugs

It has been discovered that, surprisingly, co-administration (or controlled release from a unit dosage form) of an NSAID with an adipocyte G-protein antagonist over a period of between about 4 to 12 hours provides a superior reduction of flushing in patients while reducing or eliminating symptoms of liver damage relative to previously known formulations. In an exemplary embodiment, the period of co-administration or controlled release is less than 12 hours and more than 4 hours. In another exemplary embodiment, the period is from about 5 to 9 hours. In another exemplary embodiment, the period of co-administration or controlled release is about 4, 5, 6, 7, 8, 9, 10, or 11 hours.

Thus, in another aspect, the present invention provides a pharmaceutical composition including an adipocyte G-protein antagonist and a non-steroidal anti-inflammatory drug (NSAID) in a single layer of a controlled release solid unit dosage form. The controlled release solid unit dosage may co-release the NSAID and adipocyte G-protein antagonist over a period from 4 to 12 hours. In an exemplary embodiment, the controlled release solid unit dosage form is an intermediate release solid unit dosage form (e.g. release from about less than about 12 hours and more than 4 hours, or, in some embodiments from about 5 to 9 hours). In another exemplary embodiment, the controlled release solid unit dosage form may co-release the NSAID and adipocyte G-protein antagonist over a period of about 4, 5, 6, 7, 8, 9, 10, or 11 hours.

In some embodiments, the composition includes an intermediate release excipient (e.g. Methocel®, with other useful intermediate release excipients discussed in detail below in the section entitled “Pharmaceutical Compositions”). In an exemplary embodiment, the adipocyte G-protein antagonist is in powder form. Exemplary adipocyte G-protein antagonists are described above and are equally applicable here for the compositions including an adipocyte G-protein antagonist and an NSAID. Thus, in some embodiments, the adipocyte G-protein antagonist is niacin.

Although the present composition is not bound by any particular mechanism of action, there are several problems with the previously recommended Niaspan® combination therapy. First, requiring the separate ingestion of the NSAID may create problems with patients failing to adhere to the dosage schedule. Second, if the niacin and NSAID are ingested at different times, their peak presence in the blood may not coincide, which reduces the effectiveness of taking these in combination. Third, the ingestion of a higher doses of aspirin may result in undesired side effects. Therefore, the Niaspan® combination therapy is not the ideal formulation or method for treating flushing symptoms. By combining niacin and NSAID together in a pharmaceutical composition, the proper dosage is assured. Second, co-ingestion also provides substantially simultaneous peak presence in the bloodstream.

In an exemplary embodiment, an intermediate release solid unit dosage form is provided. The intermediate release solid unit dosage form includes a niacin, a nonsteroidal anti-inflammatory drug, and an intermediate release excipient. The niacin and the nonsteroidal anti-inflammatory drug are present in a single layer of the solid unit dosage. These niacin and nonsteroidal anti-inflammatory drug are provided in amounts effective to reduce flushing in a patient relative to the amount of flushing observed with niacin alone. The niacin and nonsteroidal anti-inflammatory drug may also be provided in amounts effective to increase HDL and/or HDL-2b levels. In some embodiments, the niacin and nonsteroidal anti-inflammatory drug are provided in amounts effective to at least partially inhibit a prostaglandin or cyclooxygenase action.

In another embodiment, the single layer is substantially homogeneous. The single layer may be formed by thoroughly mixing the niacin and the nonsteroidal anti-inflammatory drug. Methods of thoroughly mixing pharmaceutical agents are well known in the art and include, for example automatic mixing methods, such as electronic rotating drum mixing.

The intermediate release solid unit dosage form may further include, in addition to an NSAID and an adipocyte G-protein antagonist, an additional reagent. The additional reagent may include a PPAR-α agonist, a PPAR-γ agonist, a biguanide, and/or tryptophan. PPAR-α agonists, PPAR-γ agonists, and biguanides are discussed in detail above and are equally applicable to the compositions herein that include an adipocyte G-protein antagonist and an NSAID. Thus, in an exemplary embodiment, the intermediate release solid unit dosage additionally includes a fibrate. In a related embodiment, the fibrate is a fenofibrate.

In another exemplary embodiment, the intermediate release solid unit dosage additionally includes a biguanide. In a related embodiment, the biguanide is metformin.

In another exemplary embodiment, the intermediate release solid unit dosage additionally includes a PPAR-γ agonist. In a related embodiment, the PPAR-γ agonist is selected from rosiglitazone, pioglitazone, muraglitizone and farglitazar.

In another exemplary embodiment, the intermediate release solid unit dosage additionally includes one of the following combinations: (1) a PPAR-α agonist, a PPAR-γagonist, and a biguanide; (2) a PPAR-α agonist and a PPAR-γ agonist; (3) a fenofibrate, a rosiglitazone, and a metformin; or (4) a fenofibrate, and a pioglitazone.

In another embodiment, the invention discloses a pharmaceutical composition having a medium to low amount (relative to the normal commercially available dosages) of NSAID to avoid detrimental side effects associated with full dose NSAID administration. For example, at high doses, NSAIDs reduce a subject's ability to form bloodclots, which may be especially pronounced in the elderly. Acceptable medium to low dosages are those dosages less than 300 mg. In an exemplary embodiment, the amount of NSAID in the pharmaceutical composition is less 200 mg. In another exemplary embodiment, the NSAID amount is between about 25 mg and about 200 mg. Further acceptable dosage ranges are detailed below in the section entitled “Dosages.”

A. Non-Steroidal Anti-Inflammatory Drugs

Non-steroidal anti-inflammatory drugs (NSAIDs) at least partially inhibit the synthesis of prostaglandins, leukotrienes, and other compounds that are involved in the inflammatory process. In addition, they may protect the stomach lining, promoting blood platelet formation, inhibiting blood clotting, and regulating salt and fluid balance in the body. NSAIDs are effective in alleviating pain symptoms associated with ailments such as fever, arthritis, gout, bursitis, painful menstruation, and headache.

NSAIDS include aspirin as well as nonaspirin products. NSAIDs may be selected from: steroidal anti-inflammatory drugs including hydrocortisone and the like; antihistaminic drugs (e.g., chlorpheniramine, triprolidine); antitussive drugs (e.g., dextromethorphan, codeine, carmiphen and carbetapentane); antipruritic drugs (e.g., methidilizine and trimeprizine); anticholinergic drugs (e.g., scopolamine, atropine, homatropine, levodopa); anti-emetic and antinauseant drugs (e.g., cyclizine, meclizine, chlorpromazine, buclizine); anorexic drugs (e.g., benzphetamine, phentermine, chlorphentermine, fenfluramine); central stimulant drugs (e.g., amphetamine, methamphetamine, dextroamphetamine and methylphenidate); antiarrhythmic drugs (e.g., propanolol, procainamide, disopyraminde, quinidine, encainide); β-adrenergic blocker drugs (e.g., metoprolol, acebutolol, betaxolol, labetalol and timolol); cardiotonic drugs (e.g., milrinone, amrinone and dobutamine); antihypertensive drugs (e.g., enalapril, clonidine, hydralazine, minoxidil, guanadrel, guanethidine); diuretic drugs (e.g., amiloride and hydrochlorothiazide); vasodilator drugs (e.g., diltazem, amiodarone, isosuprine, nylidrin, tolazoline and verapamil); vasoconstrictor drugs (e.g., dihydroergotamine, ergotamine and methylsergide); antiulcer drugs (e.g., ranitidine and cimetidine); anesthetic drugs (e.g., lidocaine, bupivacaine, chlorprocaine, dibucaine); antidepressant drugs (e.g., imipramine, desipramine, amitryptiline, nortryptiline); tranquilizer and sedative drugs (e.g., chlordiazepoxide, benacytyzine, benzquinamide, flurazapam, hydroxyzine, loxapine and promazine); antipsychotic drugs (e.g., chlorprothixene, fluphenazine, haloperidol, molindone, thioridazine and trifluoperazine); antimicrobial drugs (antibacterial, antifungal, antiprotozoal and antiviral drugs); propionic acid derivatives; acetic acid derivatives; fenamic acid derivatives; biphenylcarboxylic acid derivatives; and oxicams.

In an exemplary embodiment, the intermediate release solid unit dosage includes a nonsteroidal anti-inflammatory drug selected from aspirin, ibuprofen, indomethacin, phenylbutazone, and naproxen. In another exemplary embodiment, the nonsteroidal anti-inflammatory drug is aspirin.

The term “aspirin,” as used herein includes any appropriate form of acetylsalicylic acid including buffered aspirin, enteric coated aspirin, aspirin salts such as calcium acetylsalicylate, and mixtures of aspirin with acid acceptors.

B. Tryptophan

Tryptophan is one of the twenty most common amino acids found in mammalian proteins. Tryptophan has several basic functions in the body. One of these is as a component in the biosynthesis of niacin, and subsequently of NAD/NADH, which are essential hydrogen donors for intracellular respiration. Tryptophan and niacin metabolism, like the metabolism of triglycerides, free fatty acids and methionine, all require methylation. This methylation is accomplished via methyl donors and facilitated with enzymes. When levels of niacin are high in a patient, free methyl donors are consumed in the metabolism of the excess niacin. The lack of free methyl donors which results causes an accumulation of homocysteine in the body which can lead to insulin resistance, arteriosclerotic changes, advanced renal failure, and/or increases in blood coagulation.

It is known that tryptophan can act to stabilize methylation enzymes against proteolysis in cases of elevated amounts of niacin in a patient. Therefore, the invention comprises a pharmaceutical composition comprising niacin, NSAID, and tryptophan. The invention also comprises a method of increasing HDL levels by providing a prostaglandin inhibiting amount of a pharmaceutical composition comprising niacin, NSAID, and tryptophan.

III. Methods

The compositions of the present invention (i.e. compositions including an adipocyte G-protein antagonist, PPAR-α agonist, and PPAR-γ agonist and compositions including an NSAID and adipocyte G-protein antagonist) may be used in methods to increase HDL and/or HDL-2b levels in a subject. Where the compositions include an NSAID and niacin, the components may be combined in amounts effective to decrease flushing in a subject. These methods are described in more detail below.

A. Methods of Increasing HDL and/or HDL-2b Levels

In another aspect, the compositions of the present invention may be used to increase HDL and/or HDL-2b levels in a subject. The compositions of the present invention (i.e. compositions including an adipocyte G-protein antagonist, PPAR-α agonist, and PPAR-γ agonist and compositions including an NSAID and adipocyte G-protein antagonist) are described in detail above and are equally applicable to the methods of increasing HDL and/or HDL-2b levels described herein.

In an exemplary embodiment, a method of increasing HDL levels or HDL-2b levels in a subject are provided including co-administering niacin and a nonsteroidal anti-inflammatory drug to a subject over a period of less than about 12 hours and more than about 4 hours. In a related embodiment, the period is from about 5 to 9 hours. The niacin and the nonsteroidal anti-inflammatory drug may be released from a solid unit dosage form. In some embodiments, the niacin and the nonsteroidal anti-inflammatory drug are present in a single layer of the solid unit dosage form. In a related embodiment, the single layer is substantially homogeneous, which may be formed by automatically mixing the niacin and NSAID, as described above.

Exemplary NSAID compounds and time periods for administration are described above in the section entitled “Compositions Containing an Adipocyte G-protein antagonist and a Non-Steroidal Anti-Inflammatory Drug.” Exemplary dosages are described below in the section entitled “Dosages.” The niacin and NSAID may be combined with additional reagents, including pharmaceutical excipients, as described above in the section entitled “Compositions Containing an Adipocyte G-protein Antagonist and a Non-Steroidal Anti-Inflammatory Drug.”

In another exemplary embodiment, a method is provided for treating a hyperlipidemia, dyslipidemia, atherosclerosis, a hypercholesterolemia, cardiovascular disease, diabetes, insulin resistance, and/or metabolic syndrome in a human patient in need of such treatment. The method includes administering to the patient a composition having a first amount of an adipocyte G-protein antagonist, a second amount of a PPAR-α agonist, and a third amount of a PPAR-γ agonist. The first amount, the second amount, and the third amount are together an effective amount to provide increased HDL and/or HDL-2b levels. In an exemplary embodiment, the first amount, the second amount, and the third amount are together an effective amount to provide a synergistic therapeutic HDL increasing effect, or a synergistic therapeutic HDL-2b increasing effect.

In some embodiments, the composition further includes a nonsteroidal anti-inflammatory drug. In other embodiments, the composition further includes a biguanide. The composition may also further include a pharmaceutical excipient. Exemplary adipocyte G-protein antagonists, PPAR-α agonists, PPAR-γ agonists, biguanides, NSAIDS, and combinations thereof are discussed in detail above in the section entitled “Compositions Including a Adipocyte G-protein Antagonist, a PPAR-α agonist, and a PPAR-γ agonist.” Exemplary pharmaceutical excipients are discussed in detail in the section below entitled “Pharmaceutical Excipients.” Exemplary dosages are detailed below in the section titled “Dosages.”

B. Methods of Reducing Flushing in a Patient Receiving Niacin

In another aspect, a method is provided for reducing flushing in a subject receiving niacin. The method includes co-administering the niacin and a nonsteroidal anti-inflammatory drug to the subject over a period of less than about 12 hours and more than about 4 hours. In an exemplary embodiment, the period is from about 5 to 9 hours.

The niacin and the nonsteroidal anti-inflammatory drug may be released from a solid unit dosage form. In some embodiments, the niacin and the nonsteroidal anti-inflammatory drug are present in a single layer of the solid unit dosage form. In a related embodiment, the single layer is substantially homogeneous, which may be formed by automatically mixing the niacin and NSAID, as described above.

Exemplary NSAID compounds and time periods for administration are described above in the section entitled “Compositions Containing an Adipocyte G-protein antagonist and a Non-Steroidal Anti-Inflammatory Drug.” Exemplary dosages are described below in the section entitled “Dosages.” The niacin and NSAID may be combined with additional reagents, including pharmaceutical excipients, as described above in the section entitled “Compositions Containing an Adipocyte G-protein antagonist and a Non-Steroidal Anti-Inflammatory Drug.”

IV. Assays for Testing the HDL or HDL-2b Increasing Activity

Methods of assaying for HDL and/or HDL-2b levels are well known in the art. Typically, venous blood is drawn in the morning after an overnight fast. Blood for preparation of HDL GGE analysis may be drawn into ice-cooled disodium EDTA tubes. The major lipoprotein fractions are separated by a combination of ultracentrifugation and precipitation in accordance with the Lipid Research Clinics Protocol generally known in the art. Briefly, VLDL is separated from LDL and HDL by preparative ultracentrifugation. LDL and HDL are separated by precipitation of the LDL fraction with heparin/manganese. The LDL concentration is calculated by subtraction of the HDL portion from the total concentration before precipitation. HDL-3 is separated by ultracentrifugation at a density of 1.125 kg/L and HDL-2 cholesterol is calculated by subtracting the value of HDL-3 from that of total HDL. Cholesterol and triglyceride concentrations are determined in the VLDL, LDL, and HDL fractions. In each run, the cholesterol and triglyceride analyses may be standardized against two frozen control sera of different concentrations. The control sera may be double-checked against reference methods for cholesterol and triglyceride analyses for detection of possible drift in methodology or control sera over time.

Plasma apoA-I and B concentrations may be analyzed by competitive radioimmunoassay (Pharmacia Diagnostics AB).

HDL GGE subclasses may be analyzed by a modification of the technique described by Blanche et al., Biochim Biophys Acta. 665: 408-419 (1981). In short, HDL is separated as a plasma fraction within the densities of 1.070 and 1.21 kg/L and subject to electrophoresis on polyacrylamide gradient gels (PAA 4/30, Pharmacia). The proteins are stained with amido black and scanned at wavelength 570 nm. The absorption of the gel itself is subtracted from the curves of the HDL samples. The relative areas under the curve may be assessed. The absolute concentration in milligrams of protein per milliliter for each subclass may be derived by multiplying the relative estimates for the HDL GGE subclasses by the total protein concentration of the isolated HDL fraction. The protein concentration of HDL may be analyzed according to Lowry et al. J Biol. Chem. 193: 265-275 (1951).

Alternatively, the serum sample is combined with a Direct HDL buffer so that lipoproteins other than HDL are selectively removed via a reaction with cholesterol esterase and cholesterol oxidase. Catalase is added to the buffer to remove the hydrogen peroxide by product without the formation of color. Catalase is inhibited with the addition of Direct HDL Activator and the remaining HDL cholesterol is specifically reacted with cholesterol esterase and cholesterol oxidase. In the presence of peroxidase the peroxide end product reacts with a 4-aminoantipyrine and N-(2-hydroxy-3-sulfopropyl)-3,5-dimethoxyaniline to form a colored quinine dye, which is measured spectrophotometrically at 578 nm. The procedures may be performed using Direct HDL Reagent products from Elan Pharmaceuticals in conjunction with an ATAC® 8000 Random Access Chemistry System. with an ATAC® 8000 Random Access Chemistry System.

The following references provide further exemplary methods of measuring levels of HDL and/or HDL-2b: Lipid Research Clinics Program, Manual of Laboratory Operations, Lipid and Lipoprotein analysis, DHEW Publication NIH 75-628, Bethesda Md., National Institutes of Health (1982); Warnick et al., Clin Chem 31: 217-22 (1985); Sugiuchi et al., Clin Chem 41: 717-23 (1995); Johansson et al., Arteriosclerosis, Thrombosis, and Vascular Biology. 15: 1049-1056 (1995).

V. Pharmaceutical Compositions

The compositions of the present invention (i.e. compositions including an adipocyte G-protein antagonist, PPAR-α agonist, and PPAR-γ agonist and compositions including an NSAID and adipocyte G-protein antagonist) may be provided as pharmaceutical compositions. Pharmaceutical compositions may be administered in single dosage forms that include the applicable active ingredients (e.g. niacin and an NSAID, or an adipocyte G-protein antagonist, a PPAR-α agonist, and a PPAR-γ agonist). Alternatively, the pharmaceutical composition may include multiple dosage forms, wherein each dosage form includes a different component of the applicable composition. For example, a pharmaceutical composition may include a multiple dosage form in which an adipocyte G-protein antagonist, PPAR-α agonist, and PPAR-γ agonist are provided in three different dosage forms containing one of the three components, respectively. Alternatively, the adipocyte G-protein antagonist, PPAR-α agonist, and PPAR-γ agonist may be present in a single dosage form.

A variety of dosage forms are useful in administrating the compositions of the present invention, including oral dosage forms such as tablets, capsules, pills, powders, granules, elixirs, tinctures, suspensions, syrups, and emulsions. For example, a composition including an adipocyte G-protein antagonist, PPAR-α agonist, and PPAR-γ agonist may be administered in a pharmaceutical composition that includes an adipocyte G-protein antagonist tablet, a PPAR-α agonist tablet, and a PPAR-γ agonist tablet. Each tablet dosage form may include the same or different pharmaceutical excipients and/or controlled release excipients, as described below.

The pharmaceutical preparation includes one or more unit dosage forms. The unit dosage form may be subdivided into unit doses containing appropriate quantities of the active ingredient(s). The unit dosage form can be a packaged preparation, the package containing discrete quantities of active ingredient, such as packeted tablets, capsules, powders in vials or ampoules, cachets, lozenges, or an appropriate number of any of these in packaged form. Unit dosage forms may be in a form suitable for oral, rectal, topical, intravenous injection or parenteral administration. Parenteral and intravenous forms can also include minerals and other materials to make them compatible with the type of injection or delivery system chosen.

Solid form preparations include powders, tablets, pills, capsules, cachets, suppositories, and dispersible granules. A solid unit dosage form is a unit dosage in solid form. Solid form may include solid carriers, which may also act as diluents, flavoring agents, binders, preservatives, tablet disintegrating agents, or an encapsulating material. A pharmaceutical composition of the present invention can be micronized or powdered so that it is more easily dispersed and solubilized by the body. Processes for grinding or pulverizing drugs are well known in the art, for example, by using a hammer mill or similar milling device. In powders, the carrier may be a finely divided solid, which is in a mixture with the finely divided active component. In tablets, the active ingredient may be mixed with the carrier having the necessary binding properties in suitable proportions and compacted in the shape and size desired.

Liquid form preparations include solutions, suspensions, and emulsions, for example, water or water/propylene glycol solutions. Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Compositions of the present invention may be also be administered as pharmaceutical compositions that include an intravenous (bolus or infusion), intraperitoneal, subcutaneous, and/or intramuscular dosage form.

The compositions of the present inventions may be administered in admixture with suitable pharmaceutical diluents, extenders, excipients, or carriers (collectively referred to herein as a pharmaceutically acceptable carrier or carrier materials) suitably selected with respect to the intended form of administration and as consistent with conventional pharmaceutical practices. Similarly, cachets and lozenges are included.

The pharmaceutical compositions may also be administered alone or mixed with a pharmaceutically acceptable carrier. The carrier can be a solid or liquid, and the type of carrier is generally chosen based on the type of administration being used. Exemplary carrier include lactose, agar, magnesium carbonate, magnesium stearate, talc, sugar, pectin, dextrin, starch, gelatin, tragacanth, methylcellulose, sodium carboxymethylcellulose, a low melting wax, cocoa butter, and the like. Specific examples of pharmaceutical acceptable carriers and excipients that can be used to formulate oral dosage forms of the present invention are well known to one skilled in the art. See, for example, U.S. Pat. No. 3,903,297, which is incorporated herein by reference in its entirety for all purposes.

Examples of pharmaceutical compositions useful in administering one or more components of the compositions disclosed herein are discussed, for example, in U.S. Pat. Nos. 3,845,770, 3,916,899, 4,034,758, 4,077,407, 4,777,049, 4,851,229, 4,783,337, 3,952,741, 5,178,867, 4,587,117, 4,522,625, 5,650,170 and 4,892,739, which are herein incorporated by reference in their entirety for all purposes. Further techniques and compositions for making dosage forms useful in the present invention are also well known to one skilled in the art. See, for example, 7 Modern Pharmaceutics, Chapters 9 and 10 (Banker & Rhodes, Eds., 1979); Pharmaceutical Dosage Forms: Tablets (Lieberman et al., 1981); Ansel, Introduction to Pharmaceutical Dosage Forms 2^{nd} Ed. (1976); Remington's Pharmaceutical Sciences, 17^{th} ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol. 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.), all of which are incorporated herein by reference in their entirety for all purposes.

Tablets can contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Pharmaceutical compositions may be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines.

Pharmaceutical compositions may also be coupled to soluble polymers as targetable drug carriers or as a prodrug. Suitable soluble polymers include polyvinylpyrrolidone, pyran copolymer, polyhydroxylpropylmethacrylamide-phenol, polyhydroxyethylasparta-midephenol, and polyethyleneoxide-polylysine substituted with palmitoyl residues. Furthermore, an antineoplastic mitochondrial oxidant can be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules can contain the active ingredient and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

Pharmaceutical compositions may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Pharmaceutical formulations may also include a suspending agent. Suspending agents are well known in the art and any appropriate suspending agent may be used with the compositions of the present invention. In an exemplary embodiment, the suspending agent is selected from methylcellulose and vegetable fiber, beeswax, carnauba wax, paraffin, and/or spermaceti, as well as synthetic waxes, hydrogenated vegetable oils, fatty acids, fatty alcohols and the like.

A. Kits

The present invention also includes pharmaceutical kits useful in raising HDL and/or HDL-2b levels, which include one or more containers containing a pharmaceutical composition comprising a therapeutically effective amount of a composition of the present invention. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit. It should be understood that although the specified materials and conditions are important in practicing the invention, unspecified materials and conditions are not excluded so long as they do not prevent the benefits of the invention from being realized.

B. Controlled Release Excipients

In some embodiments, the pharmaceutical formulation and/or unit dosage form(s) include a controlled time release excipient. Exemplary controlled release excipients include arabic gum, agar, alginic acid, sodium alginate, bentonite, carbomer, sodium carboxymethylcellulose, carrageenan, powdered cellulose, cetyl alcohol, dioctyl sodium sulfosuccinate, gelatin, glyceryl monostearate, hydroxyethyl cellulose, hydroxypropyl cellulose, hydroxypropyl methylcellulose, methylcellulose, octoxynol 9, oleyl alcohol, polyvinyl alcohol, povidone, propylene glycol monostearate, sodium lauryl sulfate, sorbitan esters, stearic acid, stearyl alcohol, tragacanth, and xanthan gum. In an exemplary embodiment, the controlled time release excipient is a methylcellulose. In another exemplary embodiment, the methylcellulose includes between about 40 percent and about 50 percent of the total weight of the pharmaceutical composition. Methylcelluloses may be obtained from several companies, including Dow Chemical under the trade name Methocel®.

High viscosity water-soluble 2-hydroxypropyl methyl cellulose (HPMC) may be useful in tablets and in the controlled-release tablet coating, due to its sustaining properties with respect to component release, such as niacin. High viscosity HMPC has a nominal viscosity, two percent solution, of about 100,000 CPS, methoxyl content of about 19-24, a hydroxypropyl content of about 7-12 percent, and a particle size where at least 90% passes through a USS 100 mesh screen (Methocel® K100MCR). Low viscosity HPMC may be used as the binder component of the tablet. An exemplary low viscosity HPMC has a methoxyl content of about 20-30%, a hydroxylpropyl content of about 7-12 percent, and a particle size where 100% will pass through a USS No. 30 mesh screen and 99% will pass through a USS 40 mesh screen (Methocel® EIS). In some cases, a portion of the high viscosity HPMC can be replaced by a medium viscosity HPMC, i.e., of about 2000-8,000 cps.

Useful hydrophobic components include natural and synthetic waxes such as beeswax, carnauba wax, paraffin, spermaceti, as well as synthetic waxes, hydrogenated vegetable oils, fatty acids, fatty alcohols and the like.

Coatings comprising a major portion of a polymeric material having a high degree of swelling on contact with water or other aqueous liquids may be used to further prolong the release of the an active ingredient, such as niacin, from a tablet core. Such polymers include, inter alia, cross-linked sodium carboxymethylcellulose (Acdisol-FMC), cross-linked hydroxypropylcellulose, hydroxymethylpropylcellulose, e.g., Methocel® K15M, Dow Chem. Co., carboxymethylamide, potassium methylacrylate divinylbenzene copolymer, polymethyl methacrylate, cross-linked polyvinylpyrrolidine, high molecular weight polyvinylalcohol, and the like. Hydroxypropylmethyl cellulose is available in a variety of molecular weights/viscosity grades from Dow Chemical Co. under the Methocel® designation. See also, Alderman (U.S. Pat. No. 4,704,285). These polymers may be dissolved in suitable volatile solvents, along with dyes, lubricants, flavorings and the like, and coated onto the prolonged release tablets, e.g., in amounts equal to 0.1-5% of the total tablet weight, by methods well known to the art. For example, see Remington's Pharmaceutical Sciences, A. Osol, ed., Mack Publishing Co., Easton, Pa. (16th ed. 1980) at pages 1585-1593.

Enteric coatings can also be provided to the prolonged release tablets to prevent release of the niacin until the tablet reaches the intestinal tract. Such coatings comprise mixtures of fats and fatty acids, shellac and shellac derivatives and the cellulose acid phthalates, e.g., those having a free carboxyl consent of 9-15%. See, Remington's at page 1590, and Zeitova et al. (U.S. Pat. No. 4,432,966), for descriptions of suitable enteric coating compositions.

In an exemplary embodiment, the controlled release excipient is an intermediate release excipient. An intermediate release excipient is a controlled release excipient (discussed above) that is provided in sufficient amounts to allow administration of active ingredients over a period of less than about 12 hours and more than about 4 hours. In an exemplary embodiment, the period is from about 5 to 9 hours. In some embodiments, the administration of active ingredients is from about 5 to 8 hours or from about 6 to 8 hours. In another exemplary embodiment, the administration of active ingredients is approximately 7 hours.

Tablets may include in admixture, about 5-30% high viscosity hydroxypropyl methyl cellulose, about 2-15% of a water-soluble pharmaceutical binder, about 2-20% of a hydrophobic component such as a waxy material, e.g., a fatty acid, etc.

Useful controlled release excipients for use in tablets are disclosed, for example, in U.S. Pat. Nos. 5,126,145, 5,268,181, and U.S. Pat. No. 6,596,308, which are herein incorporated by reference in their entirety for all purposes.

VI. Dosages

Exemplary dosages and ratios of components for compositions of the present invention are discussed in detail below. The dosages disclosed below are equally applicable to the pharmaceutical compositions discussed above.

A. Compositions Including an Adipocyte G-Protein Antagonist and NSAID

As discussed above, the present invention provides an intermediate release solid unit form. The intermediate release solid unit dosage form includes a niacin, a nonsteroidal anti-inflammatory drug, and an intermediate release excipient. The niacin and the nonsteroidal anti-inflammatory drug are present in a single layer of the solid unit dosage. These niacin and nonsteroidal anti-inflammatory drug are present in amounts effective to reducing flushing in a patient relative to the amount of flushing observed with niacin alone. The niacin and nonsteroidal anti-inflammatory drug may also be present in amounts effective to increase HDL and/or HDL-2b levels. In some embodiments, the niacin and nonsteroidal anti-inflammatory drug are present in amounts effective to at least partially inhibit a prostaglandin or cyclooxygenase action.

In addition, methods are provided for increasing HDL levels or HDL-2b levels in a subject are provided including co-administering niacin and a nonsteroidal anti-inflammatory drug to a subject over a period of less than 12 hours and more than 4 hours. In another exemplary embodiment, the period is from about 5 to 9 hours. The niacin and the nonsteroidal anti-inflammatory drug may be released from a solid unit dosage form. In some embodiments, the niacin and the nonsteroidal anti-inflammatory drug are present in a single layer of the solid unit dosage form. In a related embodiment, the single layer is substantially homogeneous, which may be formed by automatically mixing the niacin and NSAID, as described above.

The invention further includes a method for reducing flushing in a subject receiving niacin. The method includes co-administering the niacin and a nonsteroidal anti-inflammatory drug to the subject over a period of less than about 12 hours and more than about 4 hours. In another exemplary embodiment, the period is from about 5 to 9 hours.

The specific dosage of an NSAID described herein are exemplified by dosages of aspirin. However, one skilled in that art will recognize that, based on these examples, dosages of other NSAIDs may be determined. In an exemplary embodiment, the dosage of aspirin provided in the intermediate release solid unit form, administered in the methods for increasing HDL levels or HDL-2b levels in a subject, and administered in the methods for reducing flushing is from about 25 to 1000 mg. In another exemplary embodiment, the amount of aspirin is from about 25 to 450 mg. In another exemplary embodiment, the amount of aspirin is from about 160 to 450 mg. In another exemplary embodiment, the amount of aspirin is from about 165 to 450 mg. In another exemplary embodiment, the amount of aspirin is from about 170 to 450 mg. In another exemplary embodiment, the amount of aspirin is from about 50 to 2 g. In another exemplary embodiment, the amount of aspirin is from about 60 to 800 mg. In another exemplary embodiment, the amount of aspirin is from about 60 to 100 mg. In an exemplary embodiment, the aspirin is aspirin.

In an exemplary embodiment, the dosage of niacin administered in the intermediate release solid unit form, the methods for increasing HDL levels or HDL-2b levels in a subject, and the methods for reducing flushing is from about 20 to 2000 mg. In another exemplary embodiment, the amount of niacin is from about 50 to 2000 mg. In another exemplary embodiment, the amount of niacin is from about 50 to 1000 mg. In another exemplary embodiment, the amount of niacin is from about 50 to 500 mg. In another exemplary embodiment, the amount of niacin is from about 50 to 400 mg. In another exemplary embodiment, the amount of niacin is from about 50 to 375 mg. In another exemplary embodiment, the amount of niacin is from about 50 to 300 mg. In another exemplary embodiment, the amount of niacin is from about 50 to 200 mg. In another exemplary embodiment, the amount of niacin is from about 50 to 100 mg.

In some embodiments, the dosage of aspirin and/or niacin is adjusted over the course of a treatment regimen. For example, a dosage adjustment of from about 50 to 65 mg niacin with aspirin is given first as a single daily dose, and then twice a day at lunch and dinner for 1-5 weeks (e.g. approximately 3 weeks). The dose is gradually escalated to from about 100 to 125 mg niacin with aspirin then twice a day for 1-5 weeks (e.g. 3 weeks). Next, the dose is increased to about 250 mg once a day and then twice a day for three weeks. Next, the dose is again increased to about 375 mg of niacin once a day and then twice a day. An exemplary course of treatment regimen may include increasing aspirin dosages of about 41 mg, 81 mg, 161 mg, 200 mg, 250 mg, 300 mg, 325 mg, and/or 375 mg.

An exemplary course of treatment regimen for administering niacin may include increasing niacin dosages of about 62 mg (e.g. 62.5 mg), 125 mg, 250 mg, 375 mg, 500 mg, 750 mg, 1000 mg, and 2000 mg. Each dose of niacin may be provided once a day, then twice a day. Dosages may be increase over a period of time suitable to minimize flushing in a patient.

In another exemplary embodiment, a starter pack is provided that includes dosages of aspirin and niacin useful in increasing niacin dosage administration to a patient while minimizing flushing and/or liver damage. Exemplary dosages include: about 62.5 mg niacin and about 81 mg of aspirin; about 125 mg of niacin and about 161 mg of aspirin; about 250 mg of niacin and about 161 mg of aspirin; about 375 mg of niacin and about 200 mg of aspirin, about 500 mg of niacin and about 250 mg of aspirin, about 500 mg of niacin and about 325 mg of aspirin, about 750 mg niacin and about 375 mg of aspirin, and about 750 mg of niacin and about 350 mg of aspirin. Exemplary dosage mass ratios of niacin to aspirin range from about 0.77:1, to 1.5:1, to 1.8:1, to 2:1, to 2.3:1. Other exemplary dosage mass ratios ranges may be from about 3:1 to 5:1. In another exemplary embodiment, the mass ratios ranges may be from about 5:1 to 10:1.

In another exemplary embodiment, a course of administration is provided according to the following schedule:
about 1-2 weeks administering about 62.5 mg niacin and about 81 mg of aspirin every night, then twice a day after lunch and dinner for about 7 days;
about 1-2 weeks administering about 125 mg niacin and about 161 mg of aspirin every night then twice a day after lunch and dinner for about 7 days
about 1-2 weeks administering about 250 mg niacin and about 161 mg of aspirin every night then twice a day after lunch and dinner for about 7 days
about 1-2 weeks administering about 375 mg niacin and about 161 mg of aspirin every night then twice a day after lunch and dinner for about 7 days
   Maintenance dosages may subsequently be administered including up to about 750 mg of niacin and about 161 mg of aspirin not to exceed about 1125 mg of niacin in a day.

In an exemplary embodiment, the amounts of niacin and aspirin are provided in an amount that together is effective in reducing flushing in a patient. The dosages, however, may be varied depending upon the requirements of the patient, the severity of the condition being treated, and the compound being employed. Determination of the proper dosage for a particular situation is within the skill of the practitioner. Generally, treatment is initiated with smaller dosages, which are less than the optimum dose of the compound. Thereafter, the dosage is increased by small increments until the optimum effect under the circumstances is reached. For convenience, the total daily dosage may be divided and administered in portions during the day.

B. Compositions Including an Adipocyte G-Protein Antagonist, a PPAR-α Agonist, and a PPAR-γ Agonist

As discussed above, the present invention provides a composition (or pharmaceutical composition) including a first amount of an adipocyte G-protein antagonist, a second amount of a PPAR-α agonist, and a third amount of a PPAR-γ agonist. The first amount, second amount, and third amount are an effective amount to increase HDL and/or HDL-2b levels in a subject.

In addition, methods are provided for treating a hyperlipidemia, dyslipidemia, atherosclerosis, hypercholesterolemia, a cardiovascular disease, diabetes, insulin resistance, or metabolic syndrome in a human patient in need of such treatment. The method includes administering to the patient a composition having a first amount of an adipocyte G-protein antagonist, a second amount of a PPAR-α agonist, and a third amount of a PPAR-γ agonist. The first amount, the second amount, and the third amount are together an effective amount to increase HDL and/or HDL-2b levels. In an exemplary embodiment, the first amount, the second amount, and the third amount are together an effective amount to provide a synergistic therapeutic HDL increasing effect, or a synergistic therapeutic HDL-2b increasing effect.

In some embodiments, the composition further includes an NSAID. Exemplary dosage levels for the NSAID aspirin are discussed above in the context of intermediate release solid unit forms that include niacin and an NSAID and are equally applicable here. Moreover, the dosage levels discussed above in the context of niacin levels in the intermediate release solid unit forms are equally applicable here for the first amount of an adipocyte G-protein antagonist where the adipocyte G-protein antagonist is niacin. One skilled in that art will recognize that, based on these examples, dosages of other adipocyte G-protein antagonists may be determined. Likewise, the PPAR-α agonist dosages are exemplified below using dosages of fenofibrate, PPAR-γ agonist dosages are exemplified below using dosages of pioglitazone and rosiglitazone, and biguanide dosages are exemplified below using dosages of metformin. One of skilled will recognize that, based on these examples, dosages of other PPAR-α agonists, PPAR-γ agonists, and biguanides may be determined.

In an exemplary embodiment, the dosage of fenofibrate is from about 50-500 mg. In another exemplary embodiment, the dosage of fenofibrate is from about 50-350 mg. In another exemplary embodiment, the dosage of fenofibrate is from about 50 to 300 mg. In another exemplary embodiment, the dosage of fenofibrate is be selected from about 67 mg, 134 mg, 200 mg, 300 mg, and 334 mg.

In an exemplary embodiment, the dosage of pioglitazone is from about 5 to 100 mg. In another exemplary embodiment, the dosage of pioglitazone is from about 8 to 75 mg. In another exemplary embodiment, the dosage of pioglitazone is from about 10 to 50 mg. In another exemplary embodiment, the dosage of pioglitazone is selected from about 15 mg, 22.5 mg, 30 mg, or 45 mg.

In an exemplary embodiment, the dosage of rosiglitazone is from about 1 to 20 mg. In another exemplary embodiment, the dosage of rosiglitazone is from about 1-10 mg. In another exemplary embodiment, the dosage of rosiglitazone is from about 1 to 8 mg. In another exemplary embodiment, the dosage of rosiglitazone is from 2 to 8 mg. In another exemplary embodiment, the dosage of rosiglitazone selected from about 2 mg, 4 mg, and 8 mg.

In an exemplary embodiment, the dosage of metformin is from about 250 to 2000 mg. In another exemplary embodiment, the dosage of metformin is about 500 mg.

The mass ratio for adipocyte G-protein antagonist to PPAR-α agonist to PPAR-γagonist may range from about 5:3:1, to 40:6:1, to 50:30:1, to 200:30:1. Where a biguanide is employed, the mass ratios of adipocyte G-protein antagonist to PPAR-α agonist to PPAR-γagonist to biguanide may range from about 5:3:1:25 to 200:30:1:200.

The mass ratio of PPAR-α agonist to PPAR-γ agonist in the composition may range from about 1:1 to 100:1. In another exemplary embodiment, the mass ratio of PPAR-α agonist to PPAR-γ agonist in the composition ranges from about 1:1 to 50:1. In another exemplary embodiment, the mass ratio of PPAR-α agonist to PPAR-γ agonist in the composition ranges from about 2:1 to 40:1. In another exemplary embodiment, the mass ratio of PPAR-α agonist to PPAR-γ agonist in the composition ranges from about 2:1 to 30:1.

The mass ratios of PPAR-α agonist to PPAR-γ agonist in the preceding paragraphs may be combined with the following exemplary mass ratio ranges for adipocyte G-protein antagonist to PPAR-γ agonist in the composition: about 1:1 to 500:1; about 2:1 to 400:1; about 3:1 to 300:1; about 4:1 to 250:1; or about 5:1 to 200:1. In an exemplary embodiment, the adipocyte G-protein antagonist is niacin, the PPAR-α agonist is fenofibrate, and PPAR-γagonist is pioglitazone.

The mass ratios of PPAR-α agonist to PPAR-γ agonist and adipocyte G-protein antagonist to PPAR-γ in the preceding 2 paragraphs may be combined with the following exemplary mass ratio ranges for biguanide to PPAR-γ agonist in the composition: about 10:1 to 500:1; about 15:1 to 400:1; about 20:1 to 300:1; or about 25:1 to 200:1. In an exemplary embodiment, the adipocyte G-protein antagonist is niacin, the PPAR-α agonist is fenofibrate, the PPAR-γ agonist is rosiglitazone, and the biguanide is metformin.

In an exemplary embodiment, the amounts adipocyte G-protein antagonist, PPAR-α agonist, PPAR-γ agonist are provided in an amount that together is effective increasing HDL and/or HDL-2b levels. In an exemplary embodiment, the amounts adipocyte G-protein antagonist, PPAR-α agonist, PPAR-γ agonist are provided in an amount that together is effective decreasing body weight and/or body mass index (BMI) (e.g. by at least 5, 6, 7, 8, 9 or 10 pounds). The dosages, however, may be varied depending upon the requirements of the patient, the severity of the condition being treated, and the compound being employed. Determination of the proper dosage for a particular situation is within the skill of the practitioner. Generally, treatment is initiated with smaller dosages, which are less than the optimum dose of the compound. Thereafter, the dosage is increased by small increments until the optimum effect under the circumstances is reached. For convenience, the total daily dosage may be divided and administered in portions during the day.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, the features of the compositions (including pharmaceutical compositions) are equally applicable to the methods of treating disease states and/or the pharmaceutical compositions described herein. All publications, patents, and patent applications cited herein are hereby incorporated by reference in their entirety for all purposes.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

### Example 1

Intermediate release solid unit capsules were formulated by mixing together Nicotinic acid USP (niacin), Methocel E4M premium USP, Lactose NF Hydrols, and, optionally, Aspirin USP into a single layer. The ingredients were encapsulated using methods generally known in the art. The relative amounts of the ingredients for six capsules are shown in Table 2 below. For Capsules 1-5, the ingredients were mixed manually. For Capsule 6, the ingredients were mixed in a standard electric rotating drum for approximately 20-60 minutes, depending upon the amount of ingredients (e.g. about 20 minutes for amounts sufficient to form about 300 capsules and about 60 minutes for amounts sufficient to form about 1000 capsules) to form a single, homogenous layer.

TABLE 2

| | Nicotinic Acid | Aspirin | Methocel | Lactose |
|---|---|---|---|---|
| Capsule | (mg) | (mg) | (mg) | (mg) |
| 1 | 250 | 0 | 10 | 10 |
| 2 | 250 | 81 | 10 | 10 |
| 3 | 250 | 161 | 10 | 10 |
| 4 | 125 | 161 | 10 | 10 |
| 5 | 375 | 161 | 10 | 10 |
| 6 | 375 | 200 | 10 | 10 |

Capsules 1-6 were administered to ten patients and the degree of flushing was noted after administration. The results are shown in Table 3, below.

TABLE 3

| | Capsule | Capsule | Capsule | Capsule | Capsule | Capsule |
|---|---|---|---|---|---|---|
| Patient | 1 | 2 | 3 | 4 | 5 | 6 |
| 1 | − | − | − | − | + | + |
| 2 | − | − | − | − | − | + |
| 3 | − | − | + | − | − | + |
| 4 | − | − | + | + | + | + |
| 5 | − | + | − | − | + | + |
| 6 | − | − | + | + | + | + |
| 7 | − | + | + | − | + | + |
| 8 | + | + | − | + | − | + |
| 9 | − | + | + | + | + | + |
| 10 | + | + | + | + | + | + |

In Table 3, a “−” denotes moderate flushing, and a “+” denotes no flushing.

### Example 2

Marked HDL Cholesterol (HDL-C) Benefit in Patients with Low HDL-C and NIDDM and Impaired Glucose Tolerance (IGT) with Combined Treatment of Pioglitazone (PIO) and Low Dose Nicotinic Acid (NA).

Low HDL-C levels are associated with a significant risk of coronary artery disease in patients with NIDDM and impaired glucose tolerance (IGT). PIO partially reverses insulin resistance (IR) and increases HDL-C in patients with NIDDM. NA increases IR but also increases HDL-C, perhaps by another mechanism. It is postulated that by combining PIO and low dose NA there would not be an increase in insulin resistance as evidence by fasting plasma glucose (FPG) and Hg Alc and the elevation of HDL-C would be additive.

A retrospective chart review yielded 23 patients with IGT or NIDDM with a HDL<35 mg/dl and who were treated with a combination of PIO, 30 mg/d, and low dose NA (Niaspan), 500 mg/d, for 2 months. Patients were excluded if a concurrent lipid influencing medication was changed for 4 weeks before or during the period of observation. Tests of significant differences were determined by 2 tailed, paired analyses.

The baseline mean characteristics were 51.2 years of age, Alc of 5.50%, FPG of 109.4 mg/dl, and BMI of 31.0 kg/m2. Of the 23 patients, 12 were female and 9 had NIDDM. No change was observed in liver function (ALT). There were small but statistically insignificant improvement in FPG, Alc, LDL cholesterol (LDL-C), and total cholesterol (TC). Marked and highly statistical improvement (+81.5%) in HDL and somewhat less, for triglycerides (TG, −37.3%), was observed.

TABLE 4

| Lipid (mg/dl) | Baseline | Follow Up | P value |
|---|---|---|---|
| HDL-C | 28.1 | 50.1 | <0.0001 |
| LDL-C | 118 | 109 | 0.185 |
| TC | 190 | 187 | 0.671 |
| TG | 247 | 129 | 0.00212 |

Therefore, it was concluded that the combined treatment of PIO and low dose NA is associated with marked improvement in HDL-C, no evidence of increase liver toxicity and no deterioration in glycemic control. These results suggest the site of action of these agents is different and therefore are additive.

### Example 3

The following are exemplary pharmaceutical compositions of the invention:

Composition #1:
Nicotinic acid USP (niacin) 65 mg, 0.065 gm or 6.5%
Aspirin USP 41 mg, 0.041 gm or 4.1%
Methocel E4M premium USP cr grade 10 mg, 0.1 gm or 10%
Lactose NF Hydrols 10.4 mg, 0.104 gm or 10.4%
Capsule: 1-orange-opaque locking capsule

Composition #2:
Nicotinic acid USP (niacin) 125 mg, 0.125 gm or 12.5%
Aspirin USP 81 mg, 0.081 gm or 8.1%
Methocel E4M premium USP cr grade 10 mg, 0.1 gm or 10%
Lactose NF Hydrols 4 mg, 0.04 gm or 4%
Capsule: 1-powder blue-opaque locking capsule

Composition #3:
Nicotinic acid USP (niacin) 250 mg, 0.25 gm or 25%
Aspirin USP 161 mg, 0.161 gm or 16.1%
Methocel K100M USP 10.5 mg, 0.105 gm or 10.5%
Capsule: 0-orange-opaque locking capsule

Composition #4:
Nicotinic acid USP (niacin) 375 mg, 0.375 gm or 37.5%
Aspirin USP 200 mg, 0.200 gm or 20%
Methocel K100M USP 10.5 mg, 0.105 gm or 10.5%
Capsule: 0-yellow-opaque locking capsule

### Example 4

The combination of an adipocyte G-protein antagonist, a PPAR-α agonist, and a PPAR-γ agonist were administered to patients over approximately 12-28 weeks. Patients were given 3 solid unit dosage forms. The first solid unit dosage form was a capsule (“Unit 1”) including Nicotinic Acid USP (niacin), Methocel E4M premium USP, Lactose NF Hydrols, and Aspirin USP. The ingredients were combined and mixed in a standard electric rotating drum for approximately 20-60 minutes, depending upon the amount of ingredients (e.g. about 20 minutes for amounts sufficient to form about 300 capsules and about 60 minutes for amounts sufficient to form about 1000 capsules). The second solid unit dosage form (“Unit 2”) was a Lofibra® capsule or Tricor® tablet, which included 134 mg of fenofibrate for patients having a starting triglyceride level of less than 200 mg/dl, or 200 mg of fenofibrate for patients having a starting tryglyceride level of more that 200 mg/dl. The third solid unit dosage form (“Unit 3”) was Actos®, which included 30 mg of pioglitazone.

Each patient was administered Unit 1, Unit 2, and Unit 3 once a day for about 12-28 weeks. In the first week of treatment, Unit 1 included 125 mg niacin and 81 mg aspirin. In the second week of treatment, Unit 1 included 250 mg of niacin and 161 mg of aspirin. Thereafter, Unit 1 included 500 mg of niacin and 161 mg of aspirin.

Results are shown in Table 5 below.

TABLE 5

| | BMI | WT | HDL- | HDL- | HDL | HDL | LDL | | A1C | A1C |
|---|---|---|---|---|---|---|---|---|---|---|
| PATIENT | Δ | Δ | 2b Δ | 2b % | Δ | % | Δ | LDL % | Δ | % |
| 1 | 1 | 2.8 | 14 | 102 | 11 | 31.43 | −10 | −13.33 | 0 | 0.00 |
| 2 | 0.5 | 1.4 | N/A | N/A | 8 | 25.00 | 11 | 25.00 | −0.2 | −3.70 |
| 3 | 0.5 | 3 | 16 | 108 | 12 | 38.71 | −24 | −17.14 | 0 | 0.00 |
| 4 | 0 | 1 | 10 | 104 | 10 | 34.48 | −40 | −26.85 | 0.1 | 1.79 |
| 5 | 0.4 | 4 | N/A | N/A | 6 | 24.00 | 29 | 36.25 | 0.3 | −6.38 |
| 6 | 0 | 2 | N/A | N/A | 10 | 25.64 | −24 | −27.27 | −0.2 | −3.39 |
| 7 | 0.3 | 4 | N/A | N/A | 14 | 30.43 | 46 | 71.88 | −0.2 | −3.70 |
| 8 | 0 | 0.25 | N/A | N/A | 12 | 32.43 | −29 | −28.16 | 0.2 | 3.70 |
| 9 | 0.3 | 3 | N/A | N/A | 6 | 26.09 | −114 | −69.94 | 0 | 0.00 |
| 10 | 0 | 0.35 | N/A | N/A | 14 | 31.11 | 35 | 20.83 | 0.1 | 1.49 |
| 11 | 0.5 | 5 | N/A | N/A | 12 | 25.53 | −37 | −33.94 | 0.5 | 9.80 |
| 12 | 0.6 | 6 | N/A | N/A | 12 | 28.57 | −99 | −71.22 | | |
| 13 | 0.4 | 4 | N/A | N/A | 17 | 35.42 | −32 | −27.12 | 0.2 | 3.70 |
| 14 | 0.1 | 2 | N/A | N/A | 11 | 27.50 | −1 | −1.12 | 0 | 0.00 |
| 15 | 0.1 | 2 | N/A | N/A | 11 | 44.00 | 26 | 53.06 | −0.3 | −6.98 |
| 16 | 0.15 | 3 | 11 | 87 | 19 | 54.29 | −82 | −67.21 | −0.1 | −1.96 |
| 17 | 0.5 | 5 | 12 | 79 | 18 | 51.43 | −41 | −27.89 | 0 | 0.00 |
| 18 | 0.3 | 3 | 14 | 89 | 18 | 54.55 | 14 | 14.58 | 0 | 0.00 |
| 19 | 0.4 | 4 | 10 | 107 | 19 | 57.58 | 15 | 17.05 | 0.5 | 10.20 |
| 20 | 0.4 | 4 | N/A | N/A | 8 | 42.11 | 37 | 92.50 | −0.9 | −12.50 |
| 21 | 0.5 | 5 | N/A | N/A | 21 | 41.18 | 30 | 32.26 | 0 | 0.00 |
| 22 | 0.5 | 6 | 14 | 117 | 19 | 55.88 | | | −0.2 | −4.17 |
| 23 | 0.3 | 3 | N/A | N/A | 14 | 46.67 | −36 | −25.00 | 0.1 | 0.50 |
| 24 | 0.4 | 4 | 14 | 109 | 15 | 50.00 | 33 | 19.64 | 0.3 | 5.66 |
| 25 | 0.3 | 3 | N/A | N/A | 13 | 48.15 | | | −1.7 | −19.10 |
| 26 | 0 | 0 | 12 | 105 | 15 | 51.72 | −105 | −77.78 | 0 | 0.00 |
| 27 | 0 | 0 | N/A | N/A | 11 | 40.74 | −2 | −2.50 | 0 | 0.00 |
| 28 | 0 | 1 | 10 | 87 | 20 | 55.56 | −74 | −58.27 | 0.1 | 1.79 |
| 29 | 0.1 | 2 | N/A | N/A | 18 | 48.65 | −8 | −8.25 | −0.1 | −1.89 |
| 30 | 0 | 0 | 10 | 87 | 17 | 38.64 | −62 | −44.29 | 0.4 | 7.84 |
| 31 | 0 | 1 | 12 | 120 | 15 | 40.54 | | | 0 | 0.00 |
| 32 | 0.3 | 3 | 14 | 108 | 15 | 50.00 | 33 | 19.64 | 0.3 | 5.66 |
| 33 | 0.3 | 3 | 10 | 89 | 12 | 44.44 | | | −0.5 | −4.63 |
| 34 | 0.5 | 5 | 17 | 100 | 15 | 46.88 | −10 | −12.35 | 0.7 | 12.96 |
| 35 | 0 | 0 | 18 | 98 | 16 | 43.24 | −10 | −10.75 | 0 | 0.00 |
| 36 | 0.3 | 3 | 16 | 124 | 15 | 46.88 | −45 | −24.46 | −0.3 | −3.85 |
| 37 | 0.3 | 1 | 20 | 167 | 29 | 55.77 | 3 | 2.13 | 0.1 | 1.85 |
| 38 | 0 | 0 | 32 | 234 | 29 | 76.32 | −10 | −10.10 | 0 | 0.00 |
| 39 | 0 | 0 | 20 | 108 | 24 | 68.57 | −37 | −24.67 | 0 | 0.00 |
| 40 | 0 | 0 | 21 | 118 | 24 | 66.67 | −38 | −40.00 | 0.1 | 1.69 |
| 41 | 0.3 | 3 | 11 | 102 | 10 | 66.67 | −20 | −16.67 | −1.1 | −20.00 |
| 42 | 0 | 2 | 25 | 152 | 27 | 77.14 | −15 | −14.29 | 0.4 | 7.27 |
| 43 | 0.3 | 3 | 24 | 167 | 27 | 72.97 | −77 | −44.00 | 0 | 0.00 |
| 44 | 0.2 | 2 | 12 | 105 | 10 | 66.67 | −25 | −17.61 | 0.7 | 10.00 |
| 45 | 0.3 | 3 | 18 | 109 | 25 | 65.79 | | | −0.5 | −7.46 |
| 46 | 0.3 | | 23 | 134 | 26 | 76.47 | −2 | −1.46 | −0.7 | −9.86 |
| 47 | 0.25 | 3 | 21 | 120 | 24 | 63.16 | −14 | −10.94 | 0 | 0.00 |
| 48 | 0.5 | 5 | 24 | 203 | 21 | 70.00 | −30 | −22.06 | −0.7 | −12.7 |
| 49 | 0.43 | 5 | 26 | 226 | 27 | 79.41 | −60 | −30.93 | −0.4 | −6.90 |
| 50 | 0.1 | 2 | 20 | 117 | 16 | 76.19 | −22 | −19.13 | 0.5 | 8.33 |
| 51 | 0.3 | 3 | 28 | 234 | 29 | 70.73 | −87 | −48.60 | 0.1 | 1.82 |
| 52 | 0.3 | 3 | 22 | 156 | 18 | 66.67 | −18 | −12.00 | 0 | 0.00 |
| 53 | 0.8 | 8 | 36 | 402 | 44 | 176.00 | −24 | −21.62 | 0 | 0.00 |
| 54 | 0.7 | 7 | 37 | 400 | 45 | 204.55 | | | 0 | 0.00 |
| 55 | 0.3 | 6 | 34 | 380 | 45 | 118.42 | −16 | −17.20 | 0.9 | 20.45 |
| 56 | 0.5 | 5 | 25 | 208 | 23 | 92.00 | 3 | 2.38 | 0 | 0.00 |
| 57 | 0.5 | 5 | 19 | 209 | 16 | 84.21 | 32 | 20.00 | 0.5 | 8.77 |
| 58 | 0.6 | 6 | 32 | 305 | 40 | 133.33 | 24 | 24.00 | −1.7 | −26.98 |
| 59 | 0.3 | 3 | 23 | 134 | 21 | 84.00 | −64 | −50.79 | 0 | 0.00 |
| 60 | 0.3 | 3 | 20 | 123 | 20 | 80.00 | 42 | 30.22 | 0.4 | 8.00 |
| 61 | 0.5 | 5 | 25 | 182 | 28 | 127.27 | | | 0 | 0.00 |
| 62 | 0.5 | 5 | 28 | 217 | 38 | 92.68 | −6 | −4.20 | 0.8 | 9.64 |
| 63 | 0.4 | 4 | 21 | 170 | 22 | 100.00 | −86 | −61.87 | −0.3 | −5.77 |
| 64 | 0.6 | 6 | 28 | 250 | 34 | 94.44 | −63 | −41.72 | −0.1 | −1.45 |
| 65 | 0.5 | 5 | 26 | 231 | 34 | 82.93 | −17 | −20.24 | 0.2 | 4.26 |

In Table 5, HDL, HDL-2B, LDL and A1C are presented in mg/deciliter. A “Δ” denotes a change in the respective level before and after the course of treatment. A “%” denotes the percentage change in the respective level before and after the course of treatment. An “N/A” or blank denotes data not measured or otherwise unavailable.

It is noted that where treatment was stopped, HDL levels often decreased, Alc often gradually increased, and a weight gain was often observed within 6-12 weeks.

### Example 5

The combination of an adipocyte G-protein antagonist, PPAR-α agonist, a PPAR-γ agonist, and a biguanide were administered to patients over an average time of approximately 12 weeks. Patients were given 3 solid unit dosage forms. The first solid unit dosage form was a capsule (“Unit 1”) including Nicotinic acid USP (niacin), Methocel E4M premium USP, Lactose NF Hydrols, and Aspirin USP. The ingredients were combined and mixed in a standard electric rotating drum for approximately 20-60 minutes, depending upon the amount of ingredients (e.g. about 20 minutes for amounts sufficient to form about 300 capsules and about 60 minutes for amounts sufficient to form about 1000 capsules). The second solid unit dosage form (“Unit 2”) was a Lofibra® capsule or Tricor® tablet, which included 134 mg of fenofibrate for patients having a starting triglyceride level of less than 200 mg/dl, or 200 mg of fenofibrate for patients having a starting tryglyceride level of more that 200 mg/dl. The third solid unit dosage form (“Unit 3”) was Avandamet®, which included 2 mg of rosiglitazone and 500 mg of metformin.

Each patient was administered Unit 1 and Unit 2 once a day, and Unit 3 twice a day. In the first week of treatment, Unit 1 included 125 mg niacin and 81 mg aspirin. In the second week of treatment, Unit 1 included 250 mg of niacin and 161 mg of aspirin. Thereafter, Unit 1 included 500 mg of niacin and 161 mg of aspirin.

Results are shown in Table 6 below.

TABLE 6

| | HDL-2b | HDL-2b | | | | | | |
|---|---|---|---|---|---|---|---|---|
| Patient | Δ | % | HDL Δ | HDL % | LDL Δ | LDL % | A1C Δ | A1C % |
| A1 | 11 | 91.67 | 17 | 47.22 | −35 | −35.71 | 0.1 | 1.69 |
| A2 | 18 | 225.00 | 15 | 68.18 | −33 | −16.67 | 0.1 | 1.79 |
| A3 | 13 | 216.67 | 17 | 62.96 | −46 | −22.01 | −1.7 | −19.10 |
| A4 | 8 | 72.73 | 12 | 36.36 | 17 | 17.71 | 0 | 0.00 |
| A5 | 2 | 16.67 | 2 | 5.00 | −14 | −16.09 | −0.1 | −1.72 |
| A6 | 7 | 50.00 | 6 | 13.33 | 49 | 76.56 | −0.2 | −3.70 |
| A7 | 15 | 136.36 | 12 | 36.36 | 17 | 17.71 | 0 | 0.00 |
| A8 | 18 | 180.00 | 13 | 39.39 | 29 | 33.72 | 0.5 | 10.20 |
| A9 | 9 | 112.50 | 12 | 63.16 | 46 | 115.00 | −0.9 | −12.50 |
| A10 | 0 | 0.00 | 13 | 27.66 | 35 | 38.04 | 0 | 0.00 |
| A11 | 0 | 0.00 | 13 | 38.24 | −110 | −56.41 | −0.2 | −4.17 |
| A12 | 1 | 7.69 | 15 | 45.45 | −77 | −63.11 | −0.1 | −1.96 |
| A13 | 15 | 166.67 | 12 | 42.86 | −24 | −16.67 | −0.7 | −6.48 |
| A14 | 17 | 212.50 | 9 | 30.00 | 66 | 41.51 | 0.3 | 5.66 |
| A15 | 15 | 214.29 | 10 | 34.48 | −102 | −75.56 | 0 | 0.00 |
| A16 | 7 | 50.00 | 13 | 36.11 | −67 | −52.76 | 0.1 | 1.79 |
| A17 | −9 | −42.86 | 18 | 48.65 | −61 | −42.66 | 0 | 0.00 |
| A18 | 0 | 0.00 | 19 | 54.29 | −31 | −32.98 | 0.1 | 1.69 |
| A19 | | | 21 | 61.76 | −14 | −12.17 | 0.4 | 7.27 |
| A20 | 6 | 100.00 | 17 | 68.00 | −57 | −45.24 | 0 | 0.00 |

In Table 6, HDL, HDL-2B, LDL and A1C are presented in mg/deciliter. A “Δ” denotes a change in the respective level before and after the course of treatment. A “%” denotes the percentage change in the respective level before and after the course of treatment. A blank denotes that the data was no measured or otherwise unavailable.

It is noted that continuing treatment after the 12 week period generally resulted in additional increases in HDL and HDL-2b levels.

1. An intermediate release solid unit dosage form comprising a niacin, a nonsteroidal anti-inflammatory drug, and an intermediate release excipient, wherein the niacin and the nonsteroidal anti-inflammatory drug are present in a single layer of said solid unit dosage.

2. The intermediate release solid unit dosage form of claim 1, wherein the single layer is substantially homogeneous.

3. The intermediate release solid unit dosage form of claim 1, wherein said single layer is formed by automatically mixing the niacin and the nonsteroidal anti-inflammatory drug.

4. The intermediate release solid unit dosage form of claim 1, wherein the nonsteroidal anti-inflammatory drug is selected from the group consisting of aspirin, ibuprofen, indomethacin, phenylbutazone, and naproxen.

5. The intermediate release solid unit dosage form of claim 1, wherein the nonsteroidal anti-inflammatory drug is aspirin.

6. The intermediate release solid unit dosage form of claim 1, wherein the amount of aspirin is greater than 25 mg and no more than 450 mg.

7. The intermediate release solid unit dosage form of claim 1, wherein the amount of aspirin is greater than 160 mg and no more than 450 mg.

8. The intermediate release solid unit dosage form of claim 1, wherein the amount of aspirin is greater than 165 mg and no more than 450 mg.

9. The intermediate release solid unit dosage form of claim 1, wherein the amount of aspirin is greater than 170 mg and no more than 450 mg.

10. The intermediate release solid unit dosage form of claim 1, wherein the amount of niacin is from 50 mg to 2 g.

11. The intermediate release solid unit dosage form of claim 1, wherein the amount of niacin is from 60 mg to 800 mg.

12. The intermediate release solid unit dosage form of claim 1, wherein the amount of niacin is from 60 mg to 100 mg.

13. The intermediate release solid unit dosage form of claim 1, wherein the mass ratio of nonsteroidal anti-inflammatory to niacin is at least 1:1 and no more than 1:3.

14. The intermediate release solid unit dosage form of claim 1, further comprising a peroxisome proliferator-activated receptor-α agonist.

15. The intermediate release solid unit dosage form of claim 14, wherein said peroxisome proliferator-activated receptor-α agonist is a fibrate.

16. The intermediate release solid unit dosage form of claim 14, wherein said peroxisome proliferator-activated receptor-α agonist is a fenofibrate.

17. The intermediate release solid unit dosage form of claim 1, further comprising a biguanide.

18. The intermediate release solid unit dosage form of claim 17, wherein said biguanide is metformin.

19. The intermediate release solid unit dosage form of claim 1, further comprising a peroxisome proliferator-activated receptor-γ agonist.

20. The intermediate release solid unit dosage form of claim 19, wherein said peroxisome proliferator-activated receptor-γ agonist is a member selected from the group consisting of rosiglitazone, pioglitazone, muraglitizone and farglitazar.

21. The intermediate release solid unit dosage form of claim 1, further comprising a peroxisome proliferator-activated receptor-α agonist, a peroxisome proliferator-activated receptor-γ agonist, and a biguanide.

22. The intermediate release solid unit dosage form of claim 1, further comprising a peroxisome proliferator-activated receptor-α agonist and a peroxisome proliferator-activated receptor-γ agonist.

23. The intermediate release solid unit dosage form of claim 1, further comprising a fenofibrate, a rosiglitazone, and a metformin.

24. The intermediate release solid unit dosage form of claim 1, further comprising a fenofibrate, and a pioglitazone.

25. A method of increasing HDL levels or HDL-2b levels in a subject comprising co-administering niacin and a nonsteroidal anti-inflammatory drug to a subject over a period of less than 12 hours and more than 4 hours.

26. The method of claim 25, wherein said niacin and said nonsteroidal anti-inflammatory drug are released from a solid unit dosage form.

27. The method of claim 26, wherein the niacin and the nonsteroidal anti-inflammatory drug are present in a single layer of said solid unit dosage form.

28. The method of claim 27, wherein the single layer is substantially homogeneous.

29. The method of claim 25, wherein the nonsteroidal anti-inflammatory drug is selected from the group consisting of aspirin, ibuprofen, indomethacin, phenylbutazone, and naproxen.

30. The method of claim 25, wherein the nonsteroidal anti-inflammatory drug is aspirin.

31. The method of claim 25, wherein the amount of aspirin is greater than 25 mg and no more than 450 mg.

32. The method of claim 25, wherein the amount of aspirin is greater than 160 mg and no more than 450 mg.

33. The method of claim 25, wherein the amount of aspirin is greater than 165 mg and no more than 450 mg.

34. The method of claim 25, wherein the amount of aspirin is greater than 170 mg and no more than 450 mg.

35. The method of claim 25, wherein the amount of niacin is from 50 mg to 2 g.

36. The method of claim 25, wherein the amount of niacin is from 60 mg to 800 mg.

37. The method of claim 25, wherein the amount of niacin is from 60 mg to 100 mg.

38. The method of claim 25, wherein the mass ratio of nonsteroidal anti-inflammatory to niacin is at least 1:1 and no more than 1:3.

39. The method of claim 25, further comprising administering a peroxisome proliferator-activated receptor-α agonist.

40. The method of claim 39, wherein said peroxisome proliferator-activated receptor-α agonist is a fibrate.

41. The method of claim 39, wherein said peroxisome proliferator-activated receptor-α agonist is a fenofibrate.

42. The method of claim 25, further comprising a biguanide.

43. The method of claim 42, wherein said biguanide is metformin.

44. The method of claim 25, further comprising a peroxisome proliferator-activated receptor-γ agonist.

45. The method of claim 44, wherein said peroxisome proliferator-activated receptor-γ agonist is a member selected from the group consisting of rosiglitazone, pioglitazone, muraglitizone and farglitazar.

46. The method of claim 25, further comprising a peroxisome proliferator-activated receptor-α agonist, a peroxisome proliferator-activated receptor-γ agonist, and a biguanide.

47. The method of claim 25, further comprising a peroxisome proliferator-activated receptor-α agonist and a peroxisome proliferator-activated receptor-γ agonist.

48. The method of claim 25, further comprising a fenofibrate, a rosiglitazone, and a metformin.

49. The method of claim 25, further comprising a fenofibrate, and a pioglitazone.

50. A method of reducing flushing in a subject receiving niacin comprising co-administering said niacin and a nonsteroidal anti-inflammatory drug to the subject over a period of less than 12 hours and more than 4 hours.

51. The method of claim 50, wherein said niacin and said nonsteroidal anti-inflammatory drug are released simultaneously from a solid unit dosage form.

52. The method of claim 51, wherein the niacin and the nonsteroidal anti-inflammatory drug are present in a single layer of said solid unit dosage form.

53. The method of claim 52, wherein the single layer is substantially homogeneous.

54. The method of claim 50, wherein the nonsteroidal anti-inflammatory drug is selected from the group consisting of aspirin, ibuprofen, indomethacin, phenylbutazone, and naproxen.

55. The method of claim 50, wherein the nonsteroidal anti-inflammatory drug is aspirin.

56. The method of claim 50, wherein the amount of aspirin is greater than 25 mg and no more than 450 mg.

57. The method of claim 50, wherein the amount of aspirin is greater than 160 mg and no more than 450 mg.

58. The method of claim 50, wherein the amount of aspirin is greater than 165 mg and no more than 450 mg.

59. The method of claim 51, wherein the amount of aspirin is greater than 170 mg and no more than 450 mg.

60. The method of claim 51, wherein the amount of niacin is from 50 mg to 2 g.

61. The method of claim 51, wherein the amount of niacin is from 60 mg to 800 mg.

62. The method of claim 51, wherein the amount of niacin is from 60 mg to 100 mg.

63. The method of claim 51, wherein the mass ratio of nonsteroidal anti-inflammatory to niacin is at least 1:1 and no more than 1:3.

64. The method of claim 50, further comprising administering an additional reagent selected from the group consisting of a peroxisome proliferator-activated receptor-α agonist, a peroxisome proliferator-activated receptor-γ agonist, and a biguanide.

65. The method of claim 50, further comprising administering a peroxisome proliferator-activated receptor-α agonist.

66. The method of claim 65, wherein said peroxisome proliferator-activated receptor-α agonist is a fibrate.

67. The method of claim 65, wherein said peroxisome proliferator-activated receptor-α agonist is a fenofibrate.

68. The method of claim 50, further comprising a biguanide.

69. The method of claim 68, wherein said biguanide is metformin.

70. The method of claim 50, further comprising a peroxisome proliferator-activated receptor-γ agonist.

71. The method of claim 70, wherein said peroxisome proliferator-activated receptor-γ agonist is a member selected from the group consisting of rosiglitazone, pioglitazone, muraglitizone and farglitazar.

72. The method of claim 50, further comprising a peroxisome proliferator-activated receptor-α agonist, a peroxisome proliferator-activated receptor-γ agonist, and a biguanide.

73. The method of claim 50, further comprising a peroxisome proliferator-activated receptor-α agonist and a peroxisome proliferator-activated receptor-γ agonist.

74. The method of claim 50, further comprising a fenofibrate, a rosiglitazone, and a metformin.

75. The method of claim 50, further comprising a fenofibrate, and a pioglitazone.

76. A pharmaceutical composition comprising a first amount of an adipocyte G-protein antagonist, a second amount of a peroxisome proliferator-activated receptor-α agonist, and a third amount of a peroxisome proliferator-activated receptor-γ agonist,
wherein the first amount, the second amount, and the third amount are together an effective amount to provide a synergistic therapeutic HDL increasing effect, and/or a synergistic therapeutic HDL-2b increasing effect.

77. The composition of claim 76, wherein the first amount, the second amount, and the third amount are together an effective amount to additionally provide amelioration of cardiovascular disease.

78. The composition of claim 76, wherein the first amount, the second amount, and the third amount are together an effective amount to additionally provide a amelioration of diabetes.

79. The composition of claim 76, wherein the first amount, the second amount, and the third amount are together an effective amount to additionally provide amelioration of metabolic syndrome.

80. The composition of claim 76, wherein the first amount, the second amount, and the third amount are together an effective amount to additionally provide amelioration of hyperlipidemia.

81. The composition of claim 76, wherein the first amount, the second amount, and the third amount are together an effective amount to additionally provide amelioration of dyslipidemia.

82. The composition of claim 76, further comprising a nonsteroidal anti-inflammatory drug.

83. The composition of claim 76, wherein said peroxisome proliferator-activated receptor-α agonist is a fibrate.

84. The composition of claim 83, wherein said peroxisome proliferator-activated receptor-α agonist is a fenofibrate.

85. The composition of claim 76, further comprising a biguanide.

86. The composition of claim 85, wherein said biguanide is metformin.

87. The composition of claim 76, wherein said peroxisome proliferator-activated receptor-γ agonist is a member selected from the group consisting of rosiglitazone, pioglitazone, muraglitizone and farglitazar.

88. The composition of claim 86, wherein said adipocyte G-protein antagonist is a niacin, said peroxisome proliferator-activated receptor-α agonist is a fenofibrate, and said peroxisome proliferator-activated receptor-γ agonist is rosiglitazone.

89. The composition of claim 88, further comprising metformin.

90. The composition of claim 89, wherein the first amount is from 50 to 2000 mg, the second amount is from 30 to 300 mg, the third amount is from 1 to 10 mg, and said metformin is present in an amount from 250 to 2000 mg.

91. The composition of claim 90, further comprising aspirin in an amount from 50 to 250 mg.

92. The composition of claim 76, wherein said adipocyte G-protein antagonist is a niacin, the peroxisome proliferator-activated receptor-α agonist is a fenofibrate, and said peroxisome proliferator-activated receptor-γ agonist is pioglitazone.

93. The composition of claim 92, wherein the first amount is from 50 to 2000 mg, the second amount is from 30 to 350 mg, and the third amount is from 10 to 200 mg.

94. A method for treating a hyperlipidemia, dyslipidemia, atherosclerosis, hypercholesterolemia, cardiovascular, diabetes, insulin resistance, or metabolic syndrome in a human patient in need of such treatment, said method comprising administering to the patient a composition comprising a first amount of an adipocyte G-protein antagonist, a second amount of a peroxisome proliferator-activated receptor-α agonist, and a third amount of a peroxisome proliferator-activated receptor-γ agonist,
wherein the first amount, the second amount, and the third amount are together an effective amount to provide a synergistic therapeutic HDL increasing effect, and/or a synergistic therapeutic HDL-2b increasing effect.

95. The method of claim 94, further comprising a nonsteroidal anti-inflammatory drug.

96. The method of claim 94, wherein said peroxisome proliferator-activated receptor-α agonist is a fibrate.

97. The method of claim 96, wherein said peroxisome proliferator-activated receptor-α agonist is a fenofibrate.

98. The method of claim 94, wherein the composition further comprises a biguanide.

99. The method of claim 98, wherein said biguanide is metformin.

100. The method of claim 94, wherein said peroxisome proliferator-activated receptor-γ agonist is a member selected from the group consisting of rosiglitazone, pioglitazone, muraglitizone and farglitazar.

101. The method of claim 99, wherein said adipocyte G-protein antagonist is a niacin, said peroxisome proliferator-activated receptor-α agonist is a fenofibrate, and said peroxisome proliferator-activated receptor-γ agonist is rosiglitazone.

102. The method of claim 101, wherein said composition further comprises metformin.

103. The method of claim 102, wherein the first amount is from 50 to 2000 mg, the second amount is from 30 to 350 mg, the third amount is from 1 to 10 mg, and said metformin is present in an amount from 250 to 2000 mg.

104. The method of claim 103, further comprising aspirin in an amount from 50 to 250 mg.

105. The method of claim 94, wherein said adipocyte G-protein antagonist is a niacin, the peroxisome proliferator-activated receptor-α agonist is a fenofibrate, and said peroxisome proliferator-activated receptor-γ agonist is pioglitazone.

106. The method of claim 105, wherein the first amount is from 50 to 2000 mg, the second amount is from 30 to 300 mg, and the third amount is from 10-50 mg.
